(19) United States
(12) Patent Application Publication
(10) Pub. No.: US 2008/0063627 A1
(43) Pub. Date: Mar. 13, 2008

(54) Tissue graft materials containing biocompatible agent and methods of making and using same

(75) Inventors: Sean M. Stucke, Farmington, MN (US); Toni Michelle Heyer, St. Louis Park, MN (US)

Correspondence Address: Kagan Binder, PLLC, 221 Main Street North, Suite 200, Stillwater, MN, 55082, US

(73) Assignee: SurModics, Inc.

(21) Appl. No.: 11/519,647

(22) Filed: Sep. 12, 2006

(51) Int. Cl. A61K 35/12; C12N 5/06

(52) U.S. Cl. 424/93.7; 424/423; 435/325

(57) ABSTRACT

The invention provides implantable tissue graft materials composed of a collagenous tissue scaffold and a biocompatible agent bonded to the tissue scaffold via an activated photoreactive group. The invention further provides methods including steps of obtaining a tissue graft material comprising a collagenous tissue scaffold; contacting the collagenous tissue scaffold with a biocompatible agent composition that includes biocompatible agent and one or more photoreactive groups; and treating the collagenous tissue scaffold and biocompatible agent composition to activate the photoreactive groups and bond the biocompatible agent to the tissue scaffold via one or more activated photoreactive groups. Implantable prostheses formed of the tissue graft material are also contemplated.

### FIELD OF THE INVENTION

The invention relates to the field of tissue engineering. The invention is directed to bioengineered graft prostheses prepared from tissue material derived from animal sources. The resulting prostheses include increased biocompatible function and can be useful for implantation, repair, or use in a mammalian host.

### BACKGROUND OF THE INVENTION

The field of tissue engineering aims to develop and apply biological substitutes to restore, maintain, and/or improve tissue functions. Methods for obtaining biological tissues and tissue structures from explanted mammalian tissue, as well as processes for constructing prostheses from the tissue, have been widely investigated for surgical repair and/or for tissue or organ replacement. It is a continuing goal of researchers to develop prostheses that can successfully be used to replace or repair mammalian tissue.

Collagen is the principal structural protein in the body and constitutes approximately one-third of the total body protein. Some properties of collagen include its high tensile strength; its ion exchanging ability; its low antigenicity, due in part to masking of potential antigenic determinants by the helical structure; and its low extensibility, semipermeability, and solubility. Furthermore, collagen is a natural substance for cell adhesion. These properties and others make collagen a suitable material for tissue engineering and manufacture of implantable biological substitutes and bioremodelable prostheses.

Submucosal tissue harvested from warm-blooded vertebrates is a collagenous matrix that has shown great promise as a graft material for inducing the repair of damaged or diseased tissues in vivo, and for inducing the proliferation and differentiation of cell populations in vitro. Submucosal tissue consists primarily of extracellular matrix material prepared by mechanically removing selected portions of the mucosa and the external muscle layers and subsequently lysing resident cells with hypotonic washes. Preliminary biochemical analyses show that the composition of small intestinal submucosa is similar to that of other basement membrane/extracellular matrix structures, and consists of a complex array of collagens, proteoglycans, glycosaminoglycans, and glycoproteins. The major components commonly identified in extracellular matrix tissues similar to submucosal tissue include cell adhesion proteins, such as fibronectin, vitronectin, thrombospondin, and laminin; structural components, such as collagens and elastin; and proteoglycans, such as serglycin, versican, decorin, and perlecan.

Submucosal tissue has been shown to induce site-specific remodeling of organs and tissues. Host cells are stimulated to proliferate and differentiate into site-specific connective tissue structures, which in turn have been shown to completely replace the submucosal tissue material within a relatively short amount of time (e.g., about 90 days). The ability of submucosal tissue material to induce tissue remodeling is not completely understood, but it has been strongly associated with angiogenesis, cell migration and differentiation, and deposition of ECM.

Despite the favorable characteristics of submucosal tissue for use as a biomaterial, the surface of such tissues may cause problems with the success of medical implants fabricated of the material. The interface between host tissues and the submucosal tissue implants plays a critical role in determining the success of the implants in vivo. For example, problems associated with endothelialization and thrombogenicity of submucosal tissue implants have been noted. New grafts have been observed to cause inflammation and thrombosis, which in turn can threaten the long-term patency of the implant.

A common approach to reducing risk of inflammation and thrombosis from the submucosal tissue implant material involves fixation and processing techniques. In general, porcine small intestinal material is chemically cross-linked (for example, treated with glutaraldehyde and/or peracetic acid) to reduce immunogenicity and to allow grafting of the material from one species to another (for example, pig to human). Other physical processing techniques include gamma irradiation. The material is then commonly freeze-dried. Such chemical and/or physical processing techniques can render a membrane that is significantly altered when compared to the starting material. The collagen matrix can be distorted by compaction of the collagen fibers within collagen bundles and separation of adjacent collagen bundles. Moreover, the processed tissue can retain numerous cell remnants, which can be visualized by H&E staining.

Generally speaking, modification of biologically derived collagenous materials to generate graft prostheses aims to remove cells and cellular debris while maintaining the native collagen structure. Desirable features of graft prostheses include maintenance of the biomaterial's mechanical integrity, while generating minimal adhesions when implanted. Further, it is desirable that the biomaterial is capable of integrating into the surrounding native body tissue and becoming infiltrated with host cells, once implanted.

One approach to reducing thrombogenicity of biomaterials that has been investigated is the application of heparin to SIS. In one approach, a benzalkonium heparin (BA-heparin) isopropyl alcohol solution is applied to the prosthesis by vertically filling the lumen of an SIS prosthesis or dipping the prosthesis in the solution and then air-drying it. This procedure treats the collagen with an ionically bound BA-heparin complex. Another approach utilizes 1-ethyl-3-(3-dimethylaminopropyl)carbodiimide hydrochloride (EDC) to activate the heparin, and then to covalently bond the heparin to the collagen fiber. In yet another approach, EDC is used to activate the collagen, then covalently bond protamine to the collagen and then ionically bond heparin to the protamine.

### SUMMARY OF THE INVENTION

In some aspects, the invention provides methods for enhancing biocompatiblity of tissue graft material. The methods are accomplished by providing one or more biocompatible agents to the tissue graft material in a manner that can provide sustained biocompatible properties to the tissue graft material. In some aspects, the tissue graft material comprises a collagenous tissue scaffold. The method comprises obtaining a tissue graft material comprising a collagenous tissue scaffold; contacting the collagenous tissue scaffold with a biocompatible agent composition comprising biocompatible agent and one or more photoreactive groups; and treating the collagenous tissue scaffold and biocompatible agent composition to activate the photoreactive groups and bond the biocompatible agent to the collagenous tissue scaffold via one or more activated photoreactive groups. In accordance with the invention, the tissue graft material is obtained from a tissue of natural origin. The collagenous tissue scaffold can comprise submucosal tissue. A wide variety of biocompatible agents can be bonded to the collagenous tissue scaffold, as discussed herein. In some aspects, the photoreactive groups can be pendent from the biocompatible agent. In other aspects, the photoreactive groups can be independent of the biocompatible agent. The step of treating the collagenous tissue scaffold and biocompatible agent composition to activate the photoreactive groups can comprise irradiating the biocompatible agent composition with light in the ultraviolet or visible regions of the spectrum. The photoreactive groups can be selected as described herein.

In some method aspects, the methods comprise obtaining a tissue graft material comprising a collagenous tissue scaffold; contacting the collagenous tissue scaffold with a reagent having the formula (X)ₘ—Y-Z)ₙ where X is a photoreactive group, Y is a spacer radical, and Z is a bifunctional aliphatic acid; and treating the collagenous tissue scaffold and biocompatible agent composition to activate the photoreactive groups and bond the reagent to the collagenous tissue scaffold via one or more activated photoreactive groups. For the reagent, the values of m and n are ≧1 and while m can equal n, it is not necessary. The aliphatic acid is “bifunctional” in that it provides both an aliphatic region and an anionic (e.g., carboxylic acid) region. Once bonded to a surface, these portions cooperate in the process of attracting and binding of albumin in order to “passivate” the surface. In some embodiments, the reagent includes photoactivatible molecules having fatty acid functional groups, including polymers having multiple photoactivatible and fatty acid functional groups, as well as heterobifunctional molecules. Suitable spacers (“Y” groups) for use in preparing heterobifunctional reagents in accordance with these aspects include any di- or higher-functional spacers capable of covalently attaching a latent reactive group to an aliphatic acid in a manner that permits them both to be used for their intended purpose. The spacer may be either aliphatic or polymeric and contain various heteroatoms such as O, N, and S in place of carbon. Constituent atoms of the spacers need not be aligned linearly. Examples of suitable spacer groups include, but are not limited to, the groups consisting of substituted or unsubstituted alkylene, oxyalkylene, cycloalkylene, arylene, oxyarylene, or aralkylene groups, and having amides, ethers, and carbonates as linking functional groups to the photoreactive group, and the bifunctional aliphatic fatty acid. The spacer can also comprise a polymer that serves as a backbone. The polymer backbone can be either synthetic or naturally occurring.

In further aspects, the invention provides methods comprising obtaining a tissue graft material comprising a collagenous tissue scaffold; contacting the collagenous tissue scaffold with a reagent comprising a polymeric backbone bearing one or more pendent photoreactive groups and one or more pendent bioactive groups; and treating the collagenous tissue scaffold and biocompatible agent composition to activate the photoreactive groups and bond the reagent to the collagenous tissue scaffold via one or more activated photoreactive groups, wherein the bioactive groups are capable of specific, noncovalent interactions with complementary groups when the collagenous tissue scaffold is implanted in a patient. The bioactive agents can function by promoting the attachment of specific molecules or cells to the tissue graft material when the tissue graft material is implanted in a patient. The bioactive group can comprise a molecule having a desired specific biological activity, such as binding or enzymatic (catalytic) activity. The polymer backbone can be a natural polymer or a synthetic polymer. Suitable bioactive groups include low molecular weight bioactive groups such as cell attachment factors, growth factor, antithrombotic factors, binding receptors, ligands, enzymes, antibiotics, and nucleic acids.

The invention further contemplates implantable tissue graft material comprising a collagenous tissue scaffold and a biocompatible agent bonded to the collagenous tissue scaffold via an activated photoreactive group. The collagenous tissue scaffold can be obtained from a natural origin, as discussed herein. The tissue scaffold can comprise submucosal tissue. A wide variety of biocompatible agents can be bonded to the collagenous tissue scaffold, as discussed herein. In some aspects, the biocompatible agent is heparin or other similar biocompatible agent. The photoreactive groups can be selected as described herein. The implantable tissue graft material can be formed into an implantable prosthesis having any desired configuration, such as tubular, flat, or complex shape.

In accordance with some aspects, the invention provides methods for preparing an implantable prosthesis having enhanced biocompatible properties.

The invention further provides medical products comprising implantable prostheses provided within sterile packaging.

The invention can provide significant benefits over known techniques for preparing tissue graft materials for implantation into a mammalian host. For example, the inventive methods can be utilized in connection with tissue graft material that has been processed using a wide variety of chemical and/or physical techniques. The starting materials can therefore be selected from a wide variety of commercially available materials. In addition, the nature of the coupling between the biocompatible agent and the tissue graft material provides a stable association (e.g., covalent bond) that can enhance function of the tissue graft material within the host, and provide superior biocompatible properties in use. A covalent bond between the biocompatible agent and tissue graft material is more stable in use than an ionic bond (for example, as utilized in BA-heparin coatings on tissue such as SIS).

### BRIEF DESCRIPTION OF THE DRAWINGS

The accompanying drawings, which are incorporated in and constitute a part of this specification, illustrate several aspects of the invention and together with the description of the preferred embodiments, serve to explain the principles of the invention. A brief description of the drawings is as follows:

FIG. 1 is a graph illustrating plasminogen binding on tissue material in accordance with some aspects of the invention, wherein tissue material sample is indicated on the X-axis, and absorbance at 650 nm is illustrated on the Y-axis (nm).

### DETAILED DESCRIPTION OF THE INVENTION

The embodiments of the invention described below are not intended to be exhaustive or to limit the invention to the precise forms disclosed in the following detailed description. Rather, the embodiments are chosen and described so that others skilled in the art can appreciate and understand the principles and practices of the invention.

Throughout the specification and claims, percentages are by weight and temperatures in degrees Celsius unless otherwise indicated.

The invention relates to tissue graft material that is composed of collagenous materials and is useful in forming implantable prostheses. The formed implantable prostheses, when implanted into a mammalian host, can serve as a functioning repair, augmentation, and/or replacement tissue structure. Due to the collagenous nature of the tissue graft material, the prostheses will typically undergo controlled degradation when implanted in a mammalian host. Concomitantly with such degradation, the prostheses typically promote remodeling of surrounding tissues within the mammalian host. Thus, in some aspects, the implantable prostheses can function as a tissue replacement, and also function as a remodeling template for the ingrowth of host cells.

The tissue graft material of the invention comprises a collagenous tissue scaffold. The tissue scaffold is selected for temporary repair, replacement and/or augmentation of host tissue or structure. As such, the tissue scaffold is degradable. Suitable tissue scaffolds possess properties suitable for repair and/or replacement of host tissues when implanted, including chemical, physical and/or structural properties. Illustrative chemical properties of the tissue scaffolds include chemistry (at the surface of the tissue material as well as interior to the surface) that is suitable for cell attachment and growth when the tissue scaffold is implanted within a host. As discussed herein, tissue scaffold can include biocompatible components (such as growth factors and other cytokines) that are native to the tissue source (inherent biocompatible agents and/or activity). Illustrative physical properties include mechanical properties closely matching those of the tissue at the site of implantation. For example, when the tissue scaffold will be implanted at a location on or within the heart (an organ that is constantly in motion), the tissue scaffold can be selected to possess mechanical properties that will allow the tissue scaffold material to move with the patient heart tissue while providing the structural integrity for the desired treatment.

Regarding structural properties, the tissue scaffold comprises an open pore network that is composed of a fibrous tissue matrix. Suitable tissue scaffold can be selected to have a desirable mean pore size that is sufficient for allowing infusion of the biocompatible agent into the tissue scaffold. Suitable tissue scaffold can be selected to provide a desirable degradation rate of the overall tissue graft material. Advantageously, the porous nature of the tissue scaffolds can, in some embodiments, provide increased surface area for bonding of the biocompatible agent(s) to the tissue graft material. This increased surface area available for bonding can, in turn, allow one to utilize less overall biocompatible agent (as compared to tissue that is non-porous and therefore only capable of bonding agents at the surafce) to provide comparable biocompatible properties to the tissue graft material. In accordance with aspects of the invention, the increased surface area available for bonding can provide a more sustained biocompatible function, as compared to tissue materials that only include biocompatible agent at the surface of the tissue.

The invention relates to methods and systems for providing biocompatible properties to tissue graft materials comprising a collagenous tissue scaffold. According to the invention, a biocompatible agent is covalently coupled to a collagenous tissue scaffold to provide enhanced biocompatible properties to the collagenous tissue scaffold. In some aspects, the biocompatible agent is bonded to at least a surface of the collagenous tissue scaffold. In some aspects, the biocompatible agent is infused into the collagenous tissue scaffold and is thus bonded at more than just a surface of the collagenous tissue scaffold (for example, the biocompatible agent may be bonded to the tissue scaffold at one or more areas interior to a surface of the tissue scaffold).

In some aspects, the invention relates to methods for preparing a collagenous tissue scaffold having enhanced biocompatible properties. The enhanced biocompatible properties thus can enhance the ability of the tissue scaffold to function or exist in contact with biological fluid and/or tissue of a living organism with a net beneficial effect on the living organism. In some aspects, the enhanced biocompatible properties can provide one or more advantages, such as reduced adherence of unwanted blood components, inhibition of blood clotting, maintenance of implant surfaces free of cellular debris, controlled release of components contained within the tissue scaffold (such as growth factors), increased patient safety, reduced tendency for tissue scaffold rejection, and/or improved graft prosthesis performance.

Further, given the stable nature of the bond between the biocompatible agent and the collagenous tissue scaffold, the invention can provide sustained biocompatible properties to tissue scaffolds. The biocompatible agent is bonded to the tissue scaffold via activated photoreactive groups, as discussed herein. This results in covalent attachment to the tissue scaffold. In contrast, biocompatible agent associated with tissue graft material through ionic coupling would dissociate more readily from the tissue graft material, particularly when the biocompatible agent is hydrophilic (for example, heparin). This dissociation would be even more likely in physiologic environments with fluid flow by the implantation site, since the flow of aqueous fluid by the tissue graft material may increase likelihood of dissociation of the biocompatible agent (i.e., the hydrophilic nature of the biocompatible agent may overcome the ionic coupling of the biocompatible agent to the tissue graft material). Covalent attachment is of course more stable than simple cladding or entrapment of a tissue graft material as well, since in these cases (cladding and entrapment) there exists no chemical bond between the biocompatible agent and the tissue graft material to retain the biocompatible agent at the tissue.

Thus, in some aspects, the invention provides tissue graft material (and resulting implantable graft prostheses) capable of providing sustained biocompatible properties. In some embodiments, the biocompatible properties can be provided for a period on the order of hours to days to weeks, to months. As the tissue graft material comprising a collagenous tissue scaffold itself is broken down by the host and replaced with regenerated host tissues and cells, the biocompatible agent levels can decrease correspondingly. As the collagenous tissue scaffold degrades, biocompatible agent continues to be presented by the tissue scaffold, thereby providing an effective amount of the biocompatible agent over a treatment course to an implantation site. Generally speaking, protein can absorbed from the host blood relatively quickly in many applications (e.g., on the order of hours to days). Presence of thrombogenic factors within the tissue scaffold (such as inherent thrombogenic factors) can act to slow endothelialization down, while the presence of other factors (such as growth factors) within the tissue scaffold can increase the rate of endothelialization of the tissue graft material. In some aspects, it can be desirable to provide biocompatible properties to the tissue scaffold for a period of time sufficient to allow endothelialization of the tissue scaffold to a desired level (e.g., sufficient to reduce and/or minimize thromobgenicity of the tissue scaffold). Such period of time can be on the order of hours to days, for example, about 30 days. It will be readily appreciated that the period of time can vary widely depending upon application of the tissue graft material.

As used herein, a treatment course is a period of time during which the tissue scaffold provides a significant repair or replacement function prior to being replaced by host tissue and/or cells. The duration of the treatment course is typically determined by the application of the tissue scaffold (e.g., implantation site and function for the implantable prosthesis). Typically, a treatment course will span from hours to days to weeks or even months. For example, a typical treatment course for minimizing risk of restenosis upon implantation of a stent or stent graft is approximately 4 or more weeks. The term “implantation site” refers to the site within a patient's body at which the implantable prosthesis is placed according to the invention.

In accordance with the invention, biocompatible agent is bonded to tissue graft material comprising a collagenous tissue scaffold by contacting the collagenous tissue scaffold with a biocompatible agent composition that includes the biocompatible agent and one or more photoreactive groups. The biocompatible agent composition is then irradiated. In some embodiments, the collagenous tissue scaffold is maintained in the coating composition during the irradiation step. This can allow infusion of the biocompatible agent composition into the tissue scaffold, thereby enhancing bonding of the biocompatible agent to the collagenous tissue scaffold.

In accordance with the invention, an implantable graft prosthesis can be, for example, vascular grafts (including small diameter vascular grafts, valves, and the like), cardiac prostheses (including cardiac patches, myocardial grafts, cardiac valves), hernia repair patches, nasal septal perforation repair patches, urological repair patches, urethral slings (e.g., for urinary incontinence), wound repair prostheses (e.g., for ulcers and chronic wounds), abdominal aortic aneurysm anchors, sutures, repair patches designed to reduce adhesion (such as post-surgical adhesion), and the like. The structure of an implantable graft prosthesis can be adapted for the introduction into a mammalian host.

The invention generally relates to methods for providing biocompatible properties to a tissue graft material, in particular, collagenous tissue scaffolds. The tissue graft material that is rendered biocompatible is of a synthetic or natural material that is degradable when in contact with physiological fluids. In preferred aspects, the tissue graft material is of natural origin. The surface of the tissue graft material can be one or more surfaces of tissue graft material intended to function in contact with tissue and/or fluids of a living organism, when the tissue graft material is formed into an implantable prosthesis.

As used herein, the terms “processed collagenous tissue material” and “processed collagenous tissue matrix” mean native, normally cellular tissue that has been procured from an animal or human source, preferably a mammal. Optionally, the tissue material can be mechanically cleaned of attendant tissues; chemically cleaned of cells, cellular debris; and rendered substantially free of non-collagenous extracellular matrix components. In some aspects, the processed tissue matrix, while substantially free of cellular debris, maintains much of its native matrix structure, strength, and shape.

In accordance with the invention, the tissue graft material is obtained from a collagenous tissue source. In some aspects, the collagenous tissue comprises submucosal tissue. The submucosal tissue used as the source and starting material in accordance with the invention can comprise submucosa isolated from warm-blooded intestinal as well as other tissue sources such as the alimentary, respiratory, urinary and/or genital tracts of warm-blooded vertebrates and/or connective tissue of such vertebrates. Illustrative sources for preparing the tissue graft material of the invention are animal tissues comprising collagen, including, but not limited to: intestine, fascia lata, pericardium, dura mater, kidney, bladder, stomach, liver and other structured tissues that comprise a fibrous tissue matrix. One exemplary source for preparing the tissue graft material of the invention is an intestinal collagen layer derived from the tunica submucosa of small intestine.

Suitable sources for small intestine are mammalian organisms such as human, cow, pig, sheep, dog, goat, horse or other warm-blooded vertebrates. One illustrative source is submucosal tissue derived from pig.

An exemplary composition for preparing tissue graft material in accordance with the invention is a processed intestinal collagen layer derived from the tunica submucosa of porcine small intestine. In some embodiments, to obtain the processed intestinal collagen layer, the small intestine of a pig is harvested and attendant mesenteric tissues are grossly dissected from the intestine. The tunica submucosa can be separated, or delaminated, from the other layers of the small intestine by, for example, mechanically squeezing the raw intestinal material between opposing rollers to remove the muscular layers (tunica muscularis) and the mucosa (tunica mucosa). The tunica submucosa of the small intestine is tougher than the surrounding tissue, hence the rollers squeeze the more friable components from the submucosa.

Optionally, the submucosa may be chemically cleaned to remove debris and other substances for example, by soaking in buffer solutions at 4° C., or by soaking with sodium hydroxide (NaOH) or trypsin, or other known cleaning techniques. These cleaning techniques can be utilized, for example, to remove visibly nonapparent debris that could affect the consistency of the mechanical properties of the submucosa. Alternative means employing detergents such as TRITON X-100™ (Rohm and Haas) or sodium dodecylsulfate (SDS); enzymes such as dispase, trypsin or thermolysin; and/or chelating agents such as ethylenediaminetetracetic acid EDTA or ethylenebis(oxyethylenitrilo)tetracetic acid (EGTA) may also be included in the chemical cleaning method.

Preparation of intestinal submucosal tissue for use in accordance with the invention is also described, for example, in U.S. Pat. Nos. 4,902,508, 4,956,178, 5,554,389. To summarize, submucosal tissue is prepared from vertebrate intestine (or other organ source) by subjecting the intestinal tissue to abrasion using a longitudinal wiping motion to remove the outer layers, comprising smooth muscle tissues, and the innermost layer, i.e., at least the luminal portion of the tunica mucosa. The submucosal tissue is rinsed with saline and optionally sterilized; it can be stored in a hydrated or dehydrated state. Lyophilized or air dried submucosal tissue can be rehydrated and used in accordance with the invention without significant loss of its cell proliferative activity.

Stomach submucosa can be prepared from a segment of stomach in a procedure similar to the preparation of intestinal submucosa. A segment of stomach tissue is first subjected to abrasion using a longitudinal wiping motion to remove the outer layers (particularly the smooth muscle layers) and the luminal portions of the tunica mucosa layers. The resulting submucosa tissue can consist primarily of a cellular, eosinophilic staining (H&E staining) extracellular matrix material. See U.S. Pat. No. 6,331,319.

Liver basement membrane can be prepared by separating the membrane from the natively associated cellular components of liver tissue of a warm-blooded vertebrate. Illustrative techniques are described, for example, in U.S. Pat. No. 6,379,710. A segment of liver tissue is sliced into pieces (e.g., strips or pieces) to increase the surface area-to-volume ratio of the liver tissue. The liver tissue is then contacted with a cell-dissociation solution for a time sufficient to release cells from the matrix. The resulting liver basement membrane is rinsed one or more times with saline and optionally stored in a frozen state or a partially dehydrated state until used.

Urinary bladder submucosa and its preparation are described in U.S. Pat. No. 5,554,389. Other harvesting and separation techniques for various submucosal tissues are known and will not be described further herein.

In one illustrative embodiment, submucosal tissue for use as the source of tissue graft material of the invention includes intestinal submucosa, stomach submucosa, urinary bladder submucosa, and uterine submucosa. Intestinal submucosa is one exemplary starting material, and more particularly intestinal submucosa delaminated from both tunica muscularis and at least the tunica mucosa of warm-blooded vertebrate intestine.

In some embodiments, the processed tissue material can be treated or modified, physically and/or chemically, prior to application of a biocompatible agent in accordance with the invention. Optionally, the collagenous processed tissue material can be cross-linked or fixed. The fixation or cross-linking may be achieved by a method selected from enzymatic cross-linking, glycation, or fixation with formaldehyde, glutaraldehyde, dialdehyde starch, glyceraldehydes, cyanamide, diimides, diisocyanates, dimethyl adipimidate, carbodiimide, epoxy compounds or genepin.

Suitable enzymes for cross-linking include lysyl oxidase or a transglutaminase. A suitable transglutaminase is a tissue transglutaminase derived from pig's liver or a microbial (mTGase) derived from a variant of Streptoverticillium mobaraense. A suitable sugar for glycation is ribose.

In some aspects, the tissue may be cross-linked by carbodiimide treatment. For example, the tissue can be treated with 20 mM EDC (1-ethyl-3-3-dimethylaminopropyl carbodiimide-HCl) and 10 mM N-hydroxysuccinimide in Hepes buffer, pH 6.5 for about 72 hours.

Other chemical modifications include binding growth factors, selected extracellular matrix components, genetic material, and other agents that would affect bioremodeling and repair of the tissue being treated, repaired and/or replaced.

Physical modifications such as shaping, conditioning by stretching and relaxing, or perforating the cleaned tissue material can be performed, according to known techniques.

The processed collagenous tissue material can be decontaminated or disinfected using any conventional techniques, such as tanning with glutaraldehyde, formaldehyde tanning at acidic pH, ethylene oxide treatment, propylene oxide treatment, gamma plasma sterilization, gamma irradiation, peracetic acid sterilization, e-beam irradiation, antibiotic treatment, treatment with or any weak acid or alkali, and/or treatment with 60-80% alcohol.

The processed collagenous tissue material can be stored for use in any suitable manner. For example, processed tissue material is commonly stored in a freeze-dried state prior to use. Other storage techniques include storage in solutions of peracetic acid, glutaraldehyde, and/or antimicrobials. Other storage methods include freezing, air-drying or irradiation for storage, or storage in an air-tight container.

In some aspects, the inventive methods can be utilized in connection with tissue material that has been subjected to any one or more of the above-described processing techniques. In these aspects, the inventive methods provide great flexibility for preparing implantable graft prostheses, since a wide variety of starting materials comprising collagenous tissue can be used in accordance with the inventive techniques. Such processing as cross-linking or other modifications do not adversely impact the ability to bind biocompatible agent to the tissue graft material in accordance with the invention.

The invention generally provides methods for providing biocompatible properties to a tissue graft material. According to the invention, biocompatible agents can be selected to improve the compatibility (for example, with blood and surrounding tissues) of the tissue graft material and, in turn, implantable prostheses formed therefrom. In some aspects, the biocompatible agent, when coupled to the tissue graft material, can serve to shield the blood from the underlying tissue graft material for a desired period of time. Suitable biocompatible agents preferably reduce the likelihood for blood components to adhere to the tissue graft material and activate, thus reducing the formation of thrombus or emboli.

The biocompatible agent can be essentially any biomolecule that is attached to the surface of medical implants to improve biocompatibility of the medical implant.

In some aspects, the biocompatible agent is a biocompatible polymer. Illustrative biocompatible polymers (including peptides and proteins) having antithrombotic effects include heparin, heparin derivatives, sodium heparin, low molecular weight heparin, high affinity heparin, low affinity heparin, hirudin, polylysine, argatroban, glycoprotein IIb/IIIa platelet membrane receptor antibody, coprotein IIb/IIIa platelet membrane receptor antibody, recombinant hirudin, bivalirudin thrombin inhibitor (such as commercially available from Biogen), chondroitin sulfate, modified dextran, albumin, streptokinase, and tissue plasminogen activator (TPA). Other thrombin inhibitors include prostaglandins, forskolin, vapiprost, prostacyclin and prostacyclin analogs, PPACK-thrombin (D-phenylalanyl-L-propyl-L-arginine chloromethylketone-thrombin), dipyridamole, urokinase, nitric oxide inhibitors, and the like.

Other contemplated biocompatible polymers include fibronectin, laminin, collagen, elastin, vitronectin, tenascin, fibrinogen, thrombospondin, osteopontin, von Willibrand Factor, bone sialoprotein (and active domains thereof), or a hydrophilic polymer such as hyaluronic acid, chitosan or methyl cellulose.

Exemplary cell-cell adhesion molecules include N-cadherin and P-cadherin and active domains thereof.

Exemplary peptides include growth factors belonging to the fibroblast growth factor (FGF), epidermal growth factor (EGF), platelet-derived growth factors (PDGF), transforming growth factors (TGF), vascular endothelial growth factor (VEGF), PD-ECGF and IGF families, as well as bone morphogenic proteins (BMPs) and other bone growth factors, and neural growth factors.

Exemplary ligands or receptors include antibodies, antigens, avidin, streptavidin, biotin, protein A and protein G.

In some aspects, the biocompatible agent can be a polysaccharide, such as a natural polysaccharide. Illustrative polysaccharides include amylose, maltodextrin, amylopectin, starch, dextran, hyaluronic acid, heparin, chondroitin sulfate, dermatan sulfate, heparan sulfate, keratan sulfate, dextran sulfate, pentosan polysulfate, and chitosan. In some embodiments, low molecular weight polymers can be utilized that have little or no branching, such as those that are derived from and/or found in starch preparations, for example, amylose and maltodextrin.

In some aspects, the biocompatible agent can be conceptualized by function. In some embodiments, the biocompatible agent provides antirestenotic effects, such as anti-proliferative, anti-platelet, and/or antithrombotic effects. In some embodiments, the biocompatible agent can be selected from cell attachment factors, receptors, ligands, growth factors, enzymes, nucleic acids, and the like.

Biocompatible agents having anti-proliferative effects include, for example, angiopeptin, c-myc antisense, and the like.

Representative examples of biocompatible agents having anti-platelet effects include inhibitors of the GPIIb-IIIa platelet receptor complex, which mediates platelet aggregation. GPIIb-IIIa inhibitors can include monoclonal antibody Fab fragment c7E3, also known as abciximab (ReoPro™), and synthetic peptides or peptidomimetics such as eptifibatide (Integrilin™) or tirofiban (Agrastat™).

Exemplary antibiotics include antibiotic peptides.

In some aspects, the biocompatible polymer is present in association with the tissue graft material in an amount sufficient to provide a therapeutically useful amount of biocompatible activity to the tissue graft material. In some embodiments, the biocompatible agent is present as a coating on a surface of the tissue graft material. For example, in some aspects, the coating provides heparin activity in an amount that either prevents or reduces the accumulation of clotting factors over a period of time during which the tissue graft material (e.g., in the form of an implantable prosthesis) is used. The therapeutically useful amount can be established based upon such factors as the application of the tissue scaffold (e.g., nature of the implantation site), patient parameters, selection of bicompatible agent(s), and the like. In some aspects, the therapeutically useful amount for the inventive tissue scaffolds can be less than a therapeutically useful amount for a non-porous structure (such as a polymer catheter).

The biocompatible agent is provided in a composition for application to a tissue graft material. The biocompatible agent composition can include a solvent or dispersant and the biocompatible agent. Solvents or dispersant that can be included in the coating composition include, but are not limited to, water, alcohols (e.g., methanol, ethanol, n-propanol and isopropanol), amides (e.g., dimethylformamide, N-methylpyrrolidone), and other known solvents that would not adversely impact the tissue graft material (for example, by extracting or damaging growth factors inherently present in the tissue graft material).

In some embodiments, the biocompatible agent composition is treated to form a coated layer on a surface of the tissue graft material. As used herein, a “coating” can be composed of one or more coated layers on a surface of the tissue graft material. When describing multiple layers, reference will be made to “first coated layer,” “second coated layer,” and so on. Such reference is not meant to restrict the relative location of the coated layer on the surface of the tissue graft material (i.e., more proximally or distally from the surface of the tissue graft material), but is rather utilized to signify the distinct chemical composition of various coated layers (e.g., containing different biocompatible agents, solvents, etc.).

In some aspects, the biocompatible agent is provided at least as a coating on a surface of the tissue graft material. In some embodiments, the biocompatible agent is further infused into the tissue graft material itself, in addition to being provided at a surface of the tissue graft material. In these embodiments, the biocompatible agent can be present at locations interior to the surface of the tissue graft material as well. The porous nature of the tissue scaffold can permit such infusion and interior bonding.

The biocompatible agent is bonded to the tissue graft material via one or more activated photoreactive groups. The photoreactive groups can be included as part of the biocompatible agent (for example, pendent from the biocompatible agent) and/or can be provided as a component separate from the biocompatible agent.

In some aspects, the biocompatible agent has one or more pendent photoreactive groups. The photoreactive group can be pendent from the biocompatible agent in an amount that allows for the formation of a stable bond with the tissue graft material that provides biocompatibility, such as heparin activity. One exemplary hydrophilic biocompatible polymer with pendent photoreactive groups is photo-heparin, which is described herein. The biocompatible agent with pendent photoreactive groups can be used with other photoreactive components in the biocompatible coating composition.

In some aspects, the method of providing biocompatible properties to a tissue graft material can also include a step of contacting the tissue graft material with a second biocompatible agent, which can be different or the same as the biocompatible agent of the coating composition described, such as to provide a biocompatible agent to the tissue graft material. The second biocompatible agent can include reactive groups such as photoreactive groups. The step of contacting the tissue graft material with a second biocompatible agent can provide a coated layer (for example, a top coat) to the tissue graft material. Alternatively or in addition, the step of contacting the tissue graft material with a second biocompatible agent can provide a second biocompatible agent bonded to areas interior to the surface of the tissue graft material.

The photoreactive groups are activated and reacted to bond one or more biocompatible agent(s) to the tissue graft material. “Activated” means that the photoreactive groups have been treated with an activating source of radiation, thereby having excited the groups to an active state that resulted in bonding the groups to the tissue graft material. Use of photoreactive groups is particularly advantageous as used in the present invention for many reasons. For example, use of photoreactive groups allows the timing of bond formation to be controlled with high precision. For example, at one or more points during the coating process the photoreactive groups can be activated for a desired length of time. Use of photoreactive groups also allows one to control the extent of bond formation by controlling the amount of applied activating energy. Knowing the composition of the biocompatible agent composition and other materials associated with the tissue graft material, the use of photoreactive groups can allow bond formation between particular targets and not others. Also, a photoreactive group can be chosen to absorb activating energy at particular wavelengths and not others. This can be beneficial if the selected biocompatible agent(s) is (are) sensitive to particular wavelengths of light. The use of photoreactive groups allows a more stable bond than, for example, other associative coatings (e.g., ionic, van der Waals, and the like).

Photoreactive groups, broadly defined, are groups that respond to specific applied external light energy to undergo active specie generation with resultant covalent bonding to a target. Photoreactive groups are those groups of atoms in a molecule that retain their covalent bonds unchanged under conditions of storage but which, upon activation, form covalent bonds with other molecules. The photoreactive groups generate active species such as free radicals, nitrenes, carbenes, and excited states of ketones upon absorption of external electromagnetic or kinetic (thermal) energy. Photoreactive groups may be chosen to be responsive to various portions of the electromagnetic spectrum. Those that are responsive to the ultraviolet and visible portions of the spectrum are typically used. Photoreactive groups, including those that are described herein, are well known in the art. The present invention contemplates the use of any suitable photoreactive group for attaching the biocompatible agent to tissue graft material as described herein.

Photoreactive aryl ketones such as acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives can be used. Examples of aryl ketones include heterocyclic derivatives of anthrone, including acridone, xanthone, and thioxanthone, and their ring substituted derivatives. Some photoreactive groups include thioxanthone, and its derivatives, having excitation energies greater than about 360 nm.

These types of photoreactive groups, such as aryl ketones, are readily capable of undergoing the activation/inactivation/reactivation cycle described herein. Benzophenone is an exemplary latent reactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a tissue graft material surface or internal site within the tissue scaffold, for example), thus creating a radical pair. Subsequent collapse of the radical pair leads to formation of a new carbon-carbon bond. If a reactive bond (for example, carbon-hydrogen) is not available for bonding, the ultraviolet light-induced excitation of the benzophenone group is reversible and the molecule returns to ground state energy level upon removal of the energy source. Photoactivatible aryl ketones such as benzophenone and acetophenone are of particular importance inasmuch as these groups are subject to multiple reactivation in water and hence provide increased bonding efficiency.

The azides constitute another class of photoreactive groups and include arylazides (C₆R₅N₃) such as phenyl azide and 4-fluoro-3-nitrophenyl azide; acyl azides (—CO—N₃) such as benzoyl azide and p-methylbenzoyl azide; azido formates (—O—CO—N₃) such as ethyl azidoformate and phenyl azidoformate; sulfonyl azides (—SO₂—N₃) such as benezensulfonyl azide; and phosphoryl azides [(RO)₂PON₃] such as diphenyl phosphoryl azide and diethyl phosphoryl azide.

Diazo compounds constitute another class of photoreactive groups and include diazoalkanes (—CHN₂) such as diazomethane and diphenyldiazomethane; diazoketones (—CO—CHN₂) such as diazoacetophenone and 1-trifluoromethyl-1-diazo-2-pentanone; diazoacetates (—O—CO—CHN₂) such as t-butyl diazoacetate and phenyl diazoacetate; and beta-keto-alpha-diazoacetatoacetates (—CO—CN₂CO—O—) such as t-butyl alpha diazoacetoacetate.

Other photoreactive groups include the diazirines (—CHN₂) such as 3-trifluoromethyl-3-phenyldiazirine; and ketenes (CH═C═O) such as ketene and diphenylketene.

Photoderivatized polysaccharides, such as heparin (“photoheparin”) can be prepared by those skilled in the art as well, for example, in the manner described in U.S. Pat. No. 5,563,056 (Swan et al., see Example 4), which describes the preparation of photoheparin by reacting heparin with benzoyl-benzoyl-epsilon-aminocaproyl-N-oxysuccinimde in dimethylsulfoxide/carbonate buffer. The solvent was evaporated and the photoheparin was dialyzed against water, lyophilized, and then dissolved in water.

Other photoderivatized biocompatible agents, such as collagen, fibronectin, and laminin can be prepared as described. See, for example, U.S. Pat. No. 5,744,515 (Clapper, “Method and Implantable Article for Promoting Endothelialization”). As described in this patent, a heterobifunctional crosslinking agent can be used to photoderivatize a protein, such as a biocompatible agent. The crosslinking agent includes a benzophenone photoactivatable group on one end (benzoyl benzoic acid, BBA), a spacer in the middle (epsilon aminocaproic acid, EAC), and an amine reactive thermochemical coupling group on the other end (N-oxysuccinimide, NOS). BBA-EAC is synthesized from 4-benzoylbenzoyl chloride and 6-aminocaproic acid. Then the NOS ester of BBA-EAC is synthesized by esterifying the carboxy group of BBA-EAC by carbodiimide activation with N-hydroxysuccinimide to yield BBA-EAC-NOS. Proteins, such as collagen, fibronectin, laminin, and the like can be obtained from commercial sources. The protein is photoderivatized by adding the BBA-EAC-NOS crosslinking agent at a ratio of 10-15 moles of BBA-EAC-NOS per mole of protein.

In some aspects, the photoreactive group is provided as a component that is separate from the biocompatible agent. For example, the biocompatible agent composition can include biocompatible agent and a coupling moiety that is a photoactivatable crosslinking agent. The photoactivatable crosslinking agent can be non-ionic or ionic. The photoactivatable cross-linking agent can include at least two latent photoreactive groups that can become chemically reactive when activated (for example, exposed to an appropriate actinic energy source).

In some aspects, the coupling moiety is a non-ionic photoactivatable cross-linking agent having the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”).

In some embodiments, the coupling moiety can be an ionic photoactivatable cross-linking agent. Some ionic photoactivatable cross-linking agents are compounds having the formula: X₁—Y—X₂, wherein Y is a radical containing at least one acidic group, basic group, or a salt of an acidic group or basic group. X₁ and X₂ are each independently a radical containing a latent photoreactive group. For example, a compound of formula I can have a radical Y that contains a sulfonic acid or sulfonate group; X₁ and X₂ can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

Preferred activated photoreactive groups are selected from activated aryl ketones, for example, activated benzophenone.

In still further aspects of the invention, the biocompatible agent can be provided as a macromer. As used herein, a macromer is a polymer that is capable of undergoing further polymerization. In accordance with these aspects, the biocompatible agent as macromer includes two or more polymerizable groups. As used herein, the term “polymerizable group” generally refers to a group that is capable of propagating free radical polymerization, such as carbon-carbon double bonds. Preferred polymerizable groups include vinyl or acrylate groups. Exemplary polymerizable groups include acrylate groups, methacrylate groups, ethacrylate groups, 2-phenyl acrylate groups, itaconate groups, acrylamide groups, methacrylamide groups, and styrene groups. See, for example, U.S. Patent Publication No. US-2004-0202774-A1 (Chudzik et al., “Charged Initiator Polymers And Methods Of Use,” published Oct. 14, 2004).

Typically, polymerizable groups are incorporated into a macromer subsequent to the initial macromer formation using standard thermochemical reactions. For example, polymerizable groups can be added to collagen via reaction of amine-containing lysine residues with acryloyl chloride or glycidyl acrylate. These reactions result in collagen containing pendent polymerizable moieties. Other methods of preparing collagen macromers are described herein as well. Similarly, when synthesizing a macromer for use as described in the present invention, monomers containing reactive groups can be incorporated into the synthetic scheme. For example, hydroxyethylmethacrylate (HEMA) or aminopropylmethacrylamide (APMA) can be copolymerized with N-vinylpyrrolidone or acrylamide yielding a water-soluble polymer with pendent hydroxyl or amine groups. These pendent groups can subsequently be reacted with acryloyl chloride or glycidyl acrylate to form water-soluble polymers with pendent polymerizable groups.

For example, hyaluronic acid containing polymerizable groups has been described (see U.S. Pat. No. 6,410,044, Chudzik et al.), where hyaluronic acid was dissolved in dry formamide, and Triethylamine (TEA) and glycidyl acrylate were added to this solution. The reaction mixture was stirred at 37° C. for 82 hours. After exhaustive dialysis against deionized water using 12-14 kD molecular weight cut-off (MWCO) dialyisis tubing, the product was isolated by lyophilization. The hyaluronic acid molecules were derivatized with acrylate groups. The number and/or density of acrylate groups can be controlled using the inventive methods, for example, by controlling the relative concentration of reactive moiety to saccharide group content.

Similarly, collagen containing polymerizable groups can be accomplished in various ways. One illustrative method of preparing collagen containing polymerizable groups is described in U.S. Pat. No. 6,410,044, Chudzik et al. Collagen was dissolved in dry formamide, TEA was then added and equilibrated in ice water bath. Acryloyl chloride was added in −/25 gram aliquots. After the final addition, the solution was stirred in ice water bath for 2 hours, removed, and stirred at room temperature for 18 hours. The product was purified by dialysis against deionized water using 12-14 kD MWCO dialysis tubing, and isolated by lyophilization.

Another illustrative method of preparing collagen having polymerizable groups is as follows. Bovine Type 1 Collagen is dissolved in 0.012 N hydrochloric acid and stirred for 4 hours at 4° C. Sodium carbonate and sodium bicarbonate are added to this solution and mixed for 60 minutes at 4° C. Acrylic acid N-hydroxysuccinimide is then added, and the reaction mixture is stirred at 4° C. for 24 hours. The final product is purified by dialysis against deionized water using 12-14 kD MWCO dialysis tubing, and isolated by lyophilization.

A further illustrative method of preparing collagen macromer is described in the Examples herein.

Similar reactions can be utilized to provide polymerizable groups to other biocompatible agents, such as, but not limited to, heparin, and the like.

Hyaluronic acid, when derivatized with polymerizable groups in the manner described herein, can provide a variety of advantages. According to the invention, hyaluronic acid, as well as other polysaccharides and polyamino acids (such as collagen) can be effectively derivatized in organic, polar, anhydrous solvents and solvent combinations. One exemplary solvent is formamide, and combinations of solvents therewith. Functionally, the solvent or solvent system is one in which the polymer is sufficiently soluble and that permits its derivatization to the desired extent, while minimizing phenomena that adversely affect the biological activity of the polymer (if any), such as denaturation of collagen that adversely affects desirable cell binding.

Polymerization of the macromers can be initiated by any of the coupling moieties described here (e.g., photoactivatable crosslinking agents).

According to the invention, biocompatible agent is bonded to the tissue graft material via one or more activated photoreactive groups. The biocompatible agent is provided to the tissue graft material by contacting the tissue graft material with a biocompatible agent composition. The biocompatible agent composition includes biocompatible agent and one or more photoreactive groups. The photoreactive groups are then activated to bond the biocompatible agent to the tissue graft material. In some aspects, at least a portion of the surface of the tissue graft material is coated with the biocompatible agent composition. In some embodiments, the entire surface of the tissue graft material can be coated with a coating composition comprising biocompatible agent. The amount of the surface area provided with the coating composition can be determined according to such factors as the tissue graft material to be utilized, the application of the resulting graft prosthesis, the biocompatible agent to be utilized, mean pore size, size distribution of pores within the tissue scaffold, and the like factors. In some aspects, biocompatible agent can be bonded to areas within the tissue graft material itself. In these aspects, biocompatible agent can be bonded to areas interior to the surface. Such interior bonding can be in addition to bonding at the tissue graft material surface.

Biocompatible agent compositions described herein that include any combination biocompatible agent(s) and photoreactive group(s) can be provided to the tissue graft material, depending upon the final application of the graft prosthesis. The biocompatible agent composition can be applied to the tissue graft material using standard techniques to cover the entire surface of the material, or a portion of the tissue graft material surface. Further, the biocompatible agent composition can be disposed on the tissue graft material as a single layer or in combination with other layers. When multiple layers are provided on the surface, each individual layer can include one or more components chosen to provide a desired effect. In some embodiments, each layer is composed of the same biocompatible agent(s). Alternatively, one or more of the layers is composed of a biocompatible agent that is different from one or more of the other layers. Additionally, multiple layers of various biocompatible agents can be deposited onto the tissue graft material surface so that a particular biocompatible agent can be presented to or released from the resulting implantable prosthesis at one time.

Application techniques for bonding biocompatible agent to tissue graft material include, for example, immersion, dipping, spraying, and the like. The suitability of a biocompatible agent composition for use with a particular tissue scaffold, and in turn, the suitability of the application technique, can be evaluated by those skilled in the art, given the present description.

In some aspects, the tissue graft material is contacted by immersing the tissue graft material in the biocompatible agent composition. Without intending to be bound by a particular theory, it is believed that such immersion can allow the biocompatible agent to penetrate through the collagenous tissue scaffold in a manner that does not adversely impact inherent biocompatible agents within the collagenous tissue scaffold (natural components of the tissue, such as growth factors and other proteins). Such immersion can also allow the biocompatible agent to be bound throughout the collagenous tissue scaffold, not just at the surface of the tissue. As discussed elsewhere herein, such interior bonding of the biocompatible agent can have additional benefits. It will be understood that such penetration and/or interior bonding of the biocompatible agent can be accomplished through contacting the collagenous tissue scaffold by other methods (such as spraying, dipping and the like). Application conditions can be manipulated to allow such penetration and/or interior bonding to occur in these methods as well.

According to the invention, once the tissue graft material has been contacted with the biocompatible agent composition, the tissue graft material and biocompatible agent composition are treated to activate one or more of the photoreactive groups. In the step of treating, the photoreactive groups can be activated by irradiation using a suitable light source. “Activated” means that the photoreactive groups have been treated with an activating source of radiation, thereby having excited the groups to an active state that resulted in the bonding of the groups to the tissue graft material. Use of photoreactive groups is particularly advantageous as used in the present invention for many reasons. For example, use of photoreactive groups allows the timing of bond formation to be controlled with high precision. For example, at one or more points during the application process the photoreactive groups can be activated for a desired length of time. Use of photoreactive groups also allows one to control the extent of bond formation by controlling the amount of applied activating energy. Further, use of photoreactive groups that are coupled with the biocompatible agent allows one to prepare a biocompatible agent composition with a minimal number of components. For example, additional polymers that may not be degradable within a patient are not required to associate the biocompatible agent with the tissue graft material. Thus, upon degradation of the tissue graft material, unwanted molecules are not left at the implantation site. In some aspects, substantially all (or even the entire) implantable prosthesis is degradable.

Suitable conditions for activating the photoreactive groups can be determined, for example, based upon the tissue graft material, biocompatible agent, and photoreactive groups selected for the application. Conditions for activation include wavelength of irradiation (typically within the ultraviolet and visible portions of the spectrum), as well as duration of irradiation. Humidity can also be a factor impacting activation of the photoreactive groups. Illustrative conditions for activation include wavelength in the ultraviolet and visible portions of the spectrum (e.g., 330-340 nm) for durations in the range of about 30 seconds to about 5 minutes.

The methods of the invention provide tissue graft materials that are provided with enhanced biocompatible properties. In some aspects, the biocompatible properties are provided by one or more coated layers of biocompatible agent on a surface of the tissue graft materials. A coated layer includes photoreactive groups that have been activated and reacted to bond the biocompatible agent present in the coating to the tissue graft material.

Prior to application of biocompatible agent(s) in accordance with the inventive concepts, it is understood that the collagenous tissue scaffold may optionally retain biocompatible components (such as growth factors and other cytokines) native to the source tissue. For example, collagenous tissue scaffolds may naturally include one or more growth factors such as basic fibroblast growth factor (FGF-2), transforming growth factor beta (TGF-beta), epidermal growth factor (EGF), and/or platelet derived growth factor (PDGF). In addition, collagenous tissue scaffolds may include other biological components such as heparin, heparin sulfate, hyaluronic acid, fibronectin and the like. Thus, generally speaking, the collagenous tissue scaffolds may naturally include one or more components that induce, directly or indirectly, a cellular response such as a change in cell morphology, proliferation, growth, and/or protein or gene expression. These components can thus provide biocompatible activity that is beneficial to the host upon implantation of an implantable prosthesis composed of the collagenous tissue scaffold.

For purposes of discussion, the level of biocompatible activity that is naturally occurring in a particular collagenous tissue scaffold will be referred to as the “inherent biocompatible activity” of the collagenous tissue scaffold. The inherent biocompatible activity can be measured for the particular collagenous tissue scaffold prior to application of a biocompatible agent composition in accordance with the invention. The inherent biocompatible activity can thus establish a baseline level of the biocompatible activity, to which the inventive biocompatible tissue scaffold prepared in accordance with the invention can be compared. In some aspects, the invention provides an increased biocompatible activity relative to the inherent biocompatible activity of the collagenous tissue scaffold. In some aspects, the invention can provide a 2-fold increase, or a 5-fold increase, or 10-fold increase, or greater, relative to the inherent biocompatible activity of the collagenous tissue scaffold.

In some aspects, the increase in biocompatible activity can be represented in units. For example, in some embodiments, the invention can provide an increase in heparin activity of about 5 mU or more, or 10 mU or more, or 15 mU or more, of 20 mU or more, over the inherent heparin activity of the collagenous tissue scaffold.

In some embodiments of the invention, the tissue graft material can be provided with enhanced biocompatible properties by bonding a reagent to the tissue graft material, wherein the reagent promotes attachment of specific molecules or cells from the patient to the tissue graft material, when the tissue graft material is implanted within the patient. In accordance with these aspects, the reagent is capable of attracting biocompatible-enhancing components from the host (such as albumin) once implanted into a patient. In these embodiments, a biocompatible agent is not bonded to the tissue graft material prior to implantation; rather, biocompatible agent (e.g., albumin) is attracted to and bonded to the tissue graft material from the physiological environment of the host (e.g., the blood) after the tissue graft material is implanted within a patient. The reagent permits the binding of albumin to a surface to be enhanced. In some embodiments, the reagent comprises a bifunctional aliphatic acid. In other embodiments, the reagent comprises a polybifunctional reagent. These aspects will now be described.

In some embodiments, tissue graft material is provided with a reagent comprising a bifunctional aliphatic acid. The reagent includes a bifunctional aliphatic acid that can improve the ability of the surface to attract and bind albumin. in some aspects, the reagent is of the general formula (X)ₘ—Y-Z)ₙ where X is a photoreactive group, Y is a spacer radical, and Z is a bifunctional aliphatic acid, as each are described herein. The values of m and n are ≧1, and while m can equal n, it is not necessary. The aliphatic acid is “bifunctional” in that it provides both an aliphatic region and an anionic (e.g., carboxylic acid) region. Once bonded to a surface, these portions cooperate in the process of attracting and binding of albumin in order to “passivate” the tissue graft material.

The bifunctional aliphatic acid (“Z” group) includes both an aliphatic portion and an anionic portion. The word “aliphatic,” as used herein, refers to a substantially linear portion, e.g., a hydrocarbon backbone, capable of forming hydrophobic interactions with albumin. The word “anionic,” in turn, refers to a charged portion capable of forming further ionic interactions with the albumin molecule. By the use of a reagent of these embodiments, these portions can be covalently attached to a surface in a manner that retains their desired function, in order to attract and bind native albumin from blood and other bodily fluids.

In some embodiments, the reagent includes photoactivatible molecules having fatty acid functional groups, including polymers having multiple photoactivatible and fatty acid functional groups, as well as heterobifunctional molecules. Photoactivatible polyacrylamide copolymers containing multiple pendant fatty acid analogs and multiple pendant photoreactive groups have been synthesized from acrylamide, a benzophenone-substituted acrylamide, and N-substituted acrylamide monomers containing the fatty acid analog. Photoactivatible polyvinylpyrrolidones have also been prepared in a similar fashion. Polyacrylamide or polyvinylpyrrolidone copolymers with a single end-point photoreactive group and multiple pendant fatty acid analogs have also been synthesized. Finally, photoactivatible, heterobifunctional molecules having a benzophenone on one end and a fatty acid group on the other end optionally separated by a spacer have been made, wherein that spacer can be a hydrophobic alkyl chain or a more hydrophilic polyethylene glycol (PEG) chain.

Suitable spacers (“Y” groups) for use in preparing heterobifunctional reagents in accordance with these aspects include any di- or higher-functional spacers capable of covalently attaching a latent reactive group to an aliphatic acid in a manner that permits them both to be used for their intended purpose. Although the spacer may itself provide a desired chemical and/or physical function, preferably the spacer is non-interfering, in that it does not detrimentally affect the use of the aliphatic and ionic portions for their intended purposes. In the case of the polymeric reagents of the invention, the spacer group serves to attach the aliphatic acid to the backbone of the polymer.

The spacer may be either aliphatic or polymeric and contain various heteroatoms such as O, N, and S in place of carbon. Constituent atoms of the spacers need not be aligned linearly. For example, aromatic rings, which lack abstractable hydrogen atoms (as discussed herein), can be included as part of the spacer design in those reagents where the latent reactive group functions by initiating covalent bond formation via hydrogen atom abstraction. In its precursor form (i.e., prior to attachment of a photoreactive group and aliphatic acid), a spacer can be terminated with any suitable functionalities, such as hydroxyl, amino, carboxyl, and sulfhydryl groups, which are suitable for use in attaching a photoreactive group and the aliphatic acid by a suitable chemical reaction, e.g., conventional coupling chemistry.

Alternatively, the spacer can be formed in the course of combining a precursor containing (or capable of attaching) the photoreactive group with another containing (or capable of attaching) the aliphatic acid. For example, the aliphatic acid could be reacted with an aliphatic diamine to give an aliphatic amine derivative of the bifunctional aliphatic acid and which could be coupled with a carboxylic acid containing the photoreactive group. To those skilled in the art, it would be obvious that the photoreactive group could be attached to any appropriate thermochemical group that would react with any appropriate nucleophile containing O, N or S.

Examples of suitable spacer groups include, but are not limited to, the groups consisting of substituted or unsubstituted alkylene, oxyalkylene, cycloalkylene, arylene, oxyarylene, or aralkylene groups, and having amides, ethers, and carbonates as linking functional groups to the photoreactive group, and the bifunctional aliphatic fatty acid.

The spacer can also comprise a polymer that serves as a backbone. The polymer backbone can be either synthetic or naturally occurring. Illustrative synthetic polymers include oligomers, homopolymers, and copolymers resulting form addition or condensation polymerization. Naturally occurring polymers, such as polysaccharides, can be used as well. Preferred backbones are biologically inert, in that they do not provide a biological function that is inconsistent with, or detrimental to, their use in the manner described.

Such polymer backbones can include acrylics such as those polymerized from hydroxyethyl acrylate, hydroxyethyl methacrylate, glyceryl acrylate, glyceryl methacrylate, acrylic acid, methacrylic acid, acrylamide and methacrylamide, vinyls such as polyvinylpyrrolidone and polyvinyl alcohol; nylons such as polycaprolactam; derivatives of polylauryl lactam, polyhexamethylene adipamide and polyhexamethylene dodecanediamide, and polyurethanes; polyethers such as polyethylene oxide, polypropylene oxide and polybutylene oxide; and biodegradable polymers such as polylactic acid, polyglycolic acid, polydioxanone, polyanhydrides, and polyorthoesters.

The polymeric backbone is chosen to provide a backbone capable of bearing one or more photoreactive groups, and one or more bifunctional aliphatic acid groups. The polymeric backbone is also selected to provide a spacer between the surface and the various photoreactive groups and bifunctional aliphatic acid groups. In this manner, the reagent can be bonded to a tissue graft material or to an adjacent reagent molecule, to provide the bifunctional alphatic acid groups with sufficient freedom of movement to demonstrate optimal activity. The polymer backbones are preferably water soluble, with polyacrylamide and polyvinylpyrrolidone being particularly preferred polymers.

Reagents in accordance with these aspects can be prepared as described in U.S. Pat. Nos. 6,465,525, 6,555,587, 7,071,235 (Guire et al., “Latent Reactive Blood Compatible Agents) and related applications.

In other embodiments of the invention, the tissue graft material can be provided with a reagent comprising a polybifunctional reagent that is capable of attracting albumin once implanted into a patient. The polybifunctional reagent can comprise a polymeric backbone bearing one or more pendent photoreactive groups and one or more (and preferably two or more) pendent bioactive groups. In some aspects, the reagent includes a high molecular weight polymer backbone, preferably linear, having attached thereto an optimal density of both bioactive groups and photoreactive groups. The reagent permits useful densities of bioactive groups to be coupled to a tissue graft material surface, via one or more photoreactive groups. The backbone, in turn, can provide a spacer function of sufficient length to provide the bioactive groups with greater freedom of movement than that which could otherwise be achieved, for example, by the use of individual spacers.

In accordance with these aspects of the invention, bioactive groups can function by promoting the attachment of specific molecules or cells to the tissue graft material. Illustrative bioactive groups include, but are not limited to, proteins, peptides, carbohydrates, nucleic acids and other molecules that are capable of binding noncovalently to specific and complimentary portions of molecules or cells. Examples of such specific binding include cell surface receptors binding to ligands, antigens binding to antibodies, and enzyme substrates binding to enzymes. Preferably, the polymeric backbone comprises a synthetic polymeric backbone selected from the group consisting of addition type polymers, such as the vinyl polymers. In some exemplary embodiment, the photoreactive groups each comprise a reversibly photoactivatible ketone.

The “bioactive group” of these embodiments refers to a molecule having a desired specific biological activity, such as binding or enzymatic (catalytic) activity. The “polymer backbone” refers to a natural polymer or a synthetic polymer, for example, resulting from addition or condensation polymerization. Suitable polymer backbones include those described above with respect to the reagent comprising a bifunctional alphatic acid. Polypeptides and polyethylene glycol (PEG) are also useful as polymer backbones.

The polymeric backbone can be selected to provide a backbone capable of bearing one or more photoreactive groups and two or more bioactive groups. The polymeric backbone can also be selected to provide a spacer between the tissue graft material or to an adjacent reagent molecule, to provide the bioactive groups with sufficient freedom of movement to demonstrate optimal activity. The polymer backbones can be water soluble, with polyacrylamide and polyvinylpyrrolidone being exemplary polymers.

Suitable bioactive groups include low molecular weight bioactive groups such as cell attachment factors, growth factors, antithrombotic factors, binding receptors, ligands, enzymes, antibiotics, and nucleic acids. A reagent molecule in accordance with these embodiments can include at least one pendent bioactive group. The use of two or more pendent bioactive groups can be advantageous, since the presence of several such groups per reagent molecule tends to facilitate the use of such reagents.

Suitable cell attachment factors include attachment peptides, as well as large proteins or glycoproteins (typically 100 to 1000 kilodaltons in size) which in their native state can be firmly bound to a tissue graft material or to an adjacent cell, bind to a specific cell surface receptor, and/or bond a cell to the tissue graft material or to an adjacent cell. Naturally occurring attachment factors are primarily large molecular weight proteins, with molecular weights above 100,000 daltons.

Attachment factors bind to specific cell surface receptors, and bond cells to the tissue graft material (referred to as “substrate adhesion molecules” herein) or to adjacent cells (referred to as “cell-cell adhesion molecules” herein). See Alberts, B. et al., Molecular Biology of the Cell, 2^{nd} ed., Garland Publ., Inc., New York (1989). In addition to promoting cell attachment, each type of attachment factor can promote other cell responses, including cell migration and differentiation. Suitable attachment factors for these embodiments include substrate adhesion molecules such as the proteins laminin, fibronectin, collagens, vitronectin, tenascin, fibrinogen, thrombospondin, osteopontin, von Willibrand Factor, and bone sialoprotein. Other suitable attachment factors include cell-cell adhesion molecules (“cadherins”) such as N-cadherin and P-cadherin.

Useful attachment factors typically comprise amino acid sequences or functional analogues thereof that possess the biological activity of a specific domain of a native attachment factor, with the attachment peptide typically being about 3 to about 20 amino acids in length. Native cell attachment factors typically have one or more domains that bind to cell surface receptors and produce the cell attachment, migration, and differentiation activities of the parent molecules. These domains consist of specific amino acid sequences, several of which have been synthesized and reported to promote the attachment, spreading and/or proliferation of cells. These domains and functional analogues of these domains are termed “attachment peptides.”

Examples of attachment peptides from fibronectin include, but are not limited to, RGD (Arg-Gly-Asp) (SEQ ID NO: 1) (Kleinman, H. K., et al., Vitamins and Hormones 47:161-186, 1993), REDV (Arg-Glu-Asp-Val) (SEQ ID NO: 2) (Hubbell, J. A., et al., Ann. N.Y. Acad. Sic 665:253-258, 1992), and C/H—V (Trp-Gln-Pro-Pro-Arg-Ala-Arg-Ile) (SEQ ID NO: 3) (Mooradian, D. L. et al., Invest. Ophtl. & Vis. Sci. 34:153-164, 1993).

Examples of attachment peptides from laminin include, for example, YIGSR (Tyr-Ile-Gly-Ser-Arg) (SEQ ID NO: 4) and SIKVAV (Ser-Ile-Lys-Val-Ala-Val) (SEQ ID NO: 5) (Kleinman, H/K. et al., Vitamins and Hormones 47:161-186, 1993) and F-9 (Arg-Tyr-Val-Val-Leu-Pro-Arg-Pro-Val-Cys-Phe-Glu-Lys-Gly-Met-Asn-Tyr-Thr-Val-Arg) (SEQ ID NO: 6) (Charonis, A. S., et al., J Cell Biol. 107:1253-1260, 1988).

Illustrative attachment peptides form type IV collagen include, for example, HEP-III (Gly-Glu-Phe-Tyr-Phe-Asp-Leu-Arg-Leu-Lys-Gly-Asp-Lys) (SEQ ID NO: 7) (Koliakos, G. G., et al., J. Biol. Chem. 264:2313-2323, 1989). Desirably, attachment peptides used in these embodiments include about 3 to about 30 amino acid residues in their amino acid sequences. In some aspects, attachment peptides have no more than about 15 amino acid residues in their amino acid sequences.

Other desirable bioactive groups include growth factors, such as fibroblast growth factors, epidermal growth factor, platelet-derived growth factors, transforming growth factors, vascular endothelial growth factor, bone morphogenic proteins and other bone growth factors, neural growth factors, and the like.

Further illustrative bioactive groups include antithrombotic agents that inhibit thrombus formation or accumulation on blood contacting devices. Illustrative antithrombotic agents include heparin and hirudin (which inhibit clotting cascade proteins such as thrombin) as well as lysine. Other suitable antithrombotic agents include prostaglandins such as PGI2, PGE1 and PGDs, which inhibit platelet adhesion and activation. Still further suitable antithrombotic agents include fibrinolytic enzymes such as streptokinase, urokinase, and plasminogen activator, which degrade fibrin clots. A further suitable bioactive group consists of lysine, which binds specifially to plasminogen, which in turn can degrade fibrin clots.

Other suitable bioactive groups include binding receptors, such as antibodies and antigens. Antibodies present in connection with a tissue graft material can bind to and remove specific antigens from aqueous media that comes into contact with the immobilized antibodies. Similarly, antigens present in connection with tissue graft material can bind to and remove specific antibodies from aqueous media that comes into contact with the immobilized antigens.

Further suitable bioactive groups include receptors and their corresponding ligands. For example, avidin and streptavidin bind specifially to biotin, with avidin and streptavidin being receptors and biotin being a ligand. Similarly, fibroblastic growth factors and vascular endothelial growth factor bind with high affinity to heparin, and transforming growth factor beta and certain bone morphogenic proteins bind to type IV collagen. Also included are immunoglobulin specific binding proteins derived from bacterial sources, such as protein A and protein G, and synthetic analogues thereof.

Yet further illustrative bioactive groups include enzymes that can bind to and catalyze specific changes in substrate molecules present in aqueous media that comes into contact with the immobilized enzymes. Other desirable bioactive groups include nucleic acid sequences (e.g., DNA, RNA, and cDNA), which selectively bind complimentary nucleic acid sequences.

Additional suitable bioactive groups include antibiotics that inhibit microbial growth on biomaterial surfaces. Certain desirable antibiotics can inhibit microbial growth by binding to specific components on bacteria. A particularly desirable class of antibiotics are the antibiotic peptides that appear to inhibit microbial growth by altering the permeability of the plasma membrane via mechanisms which, at least in part, may not involve specific complimentary ligand-receptor binding (Zazloff, M., Curr. Opinion Immunol. 4:3-7, 1992).

Various parameters can be controlled to provide reagents having a desired ratio (whether on a molar or weight basis) of polymeric backbone, photoreactive groups and bioactive groups. For instance, the backbone itself will typically provide about 40 to about 400 carbon atoms per photoreactive group, or about 60 to about 300 carbon atoms.

With respect to the bioactive group, the length of the backbone can vary depending upon such factors as the size of the bioactive group and the desired coating density. For instance, for relatively small bioactive groups (MW less than about 3000), the polymeric backbone will typically be in the range of about 5 to about 200 carbon atoms per bioactive group, or in the range of about 10 to about 100. For larger bioactive groups, such as those having a molecular weight in the range of about 3000 to about 50,000, the backbone can provide, on the average, about 10 to about 5000 carbon atoms between bioactive groups, or about 50 to about 1000 carbon atoms. In each case, those skilled in the art, given the present teaching and known techniques, will be able to determine the conditions suitable to provide an optimal combination of bioactive group density and freedom of movement.

Illustrative polybifunctional reagents and methods of preparing them are described in U.S. Pat. Nos. 6,121,027 and 6,514,734 (Clapper et al., “Polybifunctional Reagent Having a Polymeric Backbone and Latent Reactive Moieties and Bioactive Groups”) and related applications.

The tissue graft material containing bonded biocompatible agent or reagent capable of promoting attachment of specific molecules or cells once implanted in a patient can be manipulated to form a tissue graft prosthesis for implantation into a mammalian host. In accordance with the invention, the tissue graft material can be manipulated to provide a prosthesis having a flat, tubular, or complex geometry. The shape of the tissue graft material will be decided by the intended application of the implantable prosthesis.

In some embodiments, the tissue graft material can be formed into a tube. The tube can be fabricated in various diameters, lengths and thickness, depending upon the indication for its use. Tubular prostheses can be used to repair or replace normally tubular structures such as vascular structures, gastrointestinal tract sections, urethra, ducts, and the like. It may also be used in nervous system repair when fabricated into a nerve growth tube packed with extracellular matrix components, growth factors, or cultured cells.

In one illustrative scheme for forming a tubular graft prosthesis, a mandrel can be chosen with a diameter measurement that will determine the diameter of the formed construct. The mandrel is preferably cylindrical or oval in cross section (depending upon the desired shape of the tubular construct and ultimate application of the implantable prosthesis) and can be made of glass, stainless steel or a nonreactive, medical grade composition. The mandrel can be straight, curved, angled, it may have branches or bifurcations, or a number of these qualities. The tissue graft material can be wrapped around the mandrel any desired number of times to form a tubular prosthesis having the desired thickness. The number of times the tissue graft material can be wrapped around the mandrel can depend upon the width of the tissue graft material sheet. For example, for a two-layer tubular construct, the width of the tissue graft material sheet would be sufficient for wrapping the sheet around the mandrel at least twice. In some embodiments, the width of the tissue graft material sheet can be slightly greater than the width that would be sufficient to wrap the sheet around the mandrel the required number of times, such that an overlapping region can be formed in the tubular prosthesis. Similarly, the length of the mandrel can dictate the length of the tube that can be formed on it. For ease of handling the construct on the mandrel, the mandrel can be longer than the length of the construct so the mandrel, and not the construct being formed, is contacted during the procedure for fabricating the tubular prosthesis.

Optionally, the mandrel can include a covering of nonreactive, medical grade quality, elastic, rubber or latex material in the form of a sleeve. While a tubular prosthesis can be formed directly on the mandrel surface, the sleeve can facilitate the removal of the formed tube from the mandrel and does not adhere to, react with, or leave residues on the tissue graft material. To remove the formed construct, the sleeve can be pulled form one end off the mandrel to carry the construct from the mandrel. This optional process can reduce stretching or otherwise stressing or risking damage to the tubular construct.

After wrapping, air bubbles, folds and creases can be smoothed out from under the material and between any biomaterial layers (when multiple layers are included).

For repair patch applications (e.g., cardiac, hernia, nasal, urological, wound, and the like), the tissue graft material can be cut to suitable dimensions for the required patch application. Other formation techniques for applications of tissue graft materials (such as SIS) are known and will not be described further here.

With reference to some specific applications, the implantable prostheses of the invention can be used to repair or replace body structures that have been damaged or diseased in host tissue. Such implantable prostheses lend themselves to a wide variety of surgical applications relating to the repair or replacement of damaged tissues, including, for example, the repair of connective tissues. Connective tissues for the purposes of the invention include bone, cartilage, muscle, tendons, ligaments, and fibrous tissue including the dermal layer of skin.

In addition, the implantable prostheses can be used in the replacement and repair of vascular, neural, dura mater, urinary bladder, and dermal tissues.

While functioning as a substitute body part or support, the implantable prosthesis can also function as a bioremodelable matrix scaffold for the ingrowth of host cells. “Bioremodeling” as used here means the production of structural collagen, vascularization, and/or cell repopulation by the ingrowth of host cells at a functional rate about equal to the rate of biodegradation, which can result in reforming and replacement of the matrix components of the implanted prosthesis by host cells and enzymes. The implantable prosthesis can retain its structural characteristics while it is remodeled by the host into all, or substantially all, host tissue, and as such, is functional as an analog of the tissue it repairs or replaces.

Tubular prostheses can be used, for example, to replace cross sections of tubular organs such as vasculature, esophagus, trachea, intestine, bowels, and fallopian tubes. These organs have a basic tubular shape with an outer surface and an inner luminal surface.

Flat sheets can be used for organ support, for example, to support prolapsed or hypermobile organs by using the sheet as a sling for the organs, such as bladder or uterus. In addition, flat sheets and tubular structures can be formed together to form a complex structure to replace or augment cardiac or venous valves.

The implantable prosthesis can be implanted to repair, augment, and/or replace diseased or damaged organs, such as abdominal wall defects, pericardium, hernias, and various other organs and structures including, but not limited to, bone, periosteum, perichondrium, intervertebral disc, articular cartilage, dermis, epidermis, bowel, ligaments, tendons, and dental structures (including dental bone and/or tissue). In addition, the tissue graft material can be used as a vascular or intra-cardiac patch, or as a replacement heart valve.

The implantable prostheses can be used in connection with vascular implants and grafts, grafts, surgical devices; synthetic prostheses; vascular prosthesis including stents, endoprosthesis, stent-graft, and endovascular-stent combinations; small diameter grafts, abdominal aortic aneurysm grafts; wound dressings and wound management devices; hemostatic barriers; mesh and hernia plugs; patches, including uterine bleeding patches, atrial septal defect (ASD) patches, patent foramen ovale (PFO) patches, ventricular septal defect (VSD) patches, pericardial patches, epicardial patches, and other generic cardiac patches; ASD, PFO, and VSD closures; percutaneous closure devices, mitral valve repair devices; heart valves, venous valves, aortic filters; venous filters; left atrial appendage filters; valve annuloplasty devices, catheters; neuroanuerysm patches; central venous access catheters, vascular access catheters, abscess drainage catheters, drug infusion catheters, parental feeding catheters, intravenous catheters (e.g., treated with antithrombotic agents), stroke therapy catheters, blood pressure and stent graft catheters; anastomosis devices and anastomotic closures; aneurysm exclusion devices; biosensors including glucose sensors; birth control devices; cosmetic implants including breast implants, lip implants, chin and cheek implants; cardiac sensors; infection control devices; membranes; tissue scaffolds; shunts including cerebral spinal fluid (CSF) shunts, glaucoma drain shunts; dental devices and dental implants; ear devices such as ear drainage tubes, tympanostomy vent tubes; ophthalmic devices; cuffs and cuff portions of devices including drainage tube cuffs, implanted drug infusion tube cuffs, catheter cuff, sewing cuff; spinal and neurological devices; nerve regeneration conduits; neurological catheters; neuropatches; orthopedic devices such as orthopedic joint implants, bone repair/augmentation devices, cartilage repair devices; urological devices and urethral devices such as urological implants, bladder devices including bladder slings, renal devices and hemodialysis devices, colostomy bag attachment devices; and biliary drainage products.

In still further aspects, the tissue graft material of the invention can be used as a cell growth substrate in a variety of forms, including a sheet-like configuration, as a coating for culture-ware to provide more physiologically relevant substrate that supports and enhances the proliferation of cells in contact with the submucosal matrix, and the like.

The invention will be further described with reference to the following non-limiting Examples.

### EXAMPLES

### Example 1

### Heparin Coating on Small Intestine Submucosal Tissue Materials

Substrate: Small intestine submucosal tissue (SIS) was obtained from Oasis Wound Matrix (Product No. 8213-1000-10, distributed by Healthpoint, Ltd. San Antonio, Tex.). The substrates were fenestrated and provided in dimensions of 7×10 cm. Substrates were stored at room temperature until use.

### Biocompatible Agent Composition:

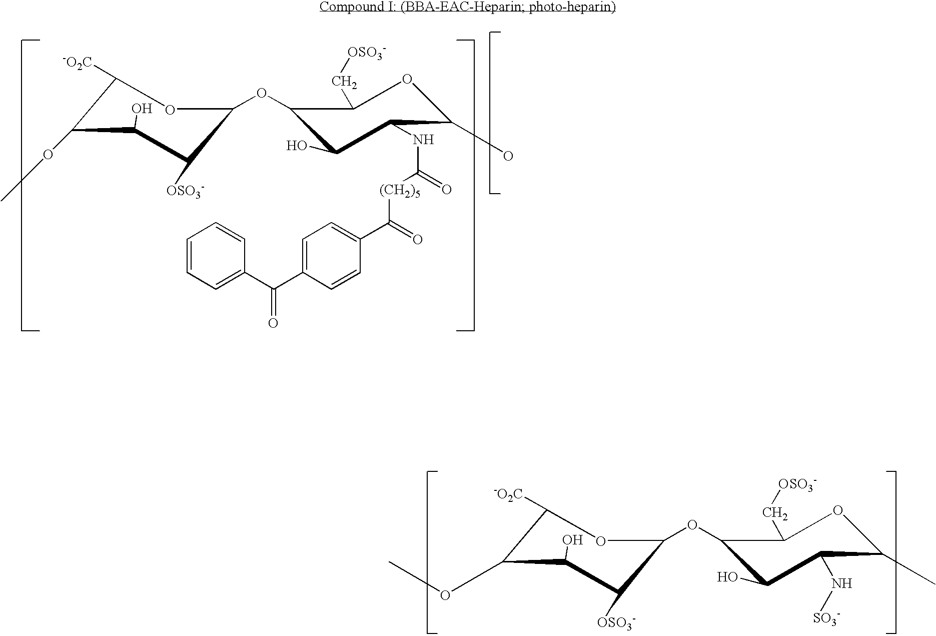

A photoreactive derivative of heparin (photoheparin) was prepared by reacting heparin with benzoyl-benzoyl-epsilon-aminocaproyl-N-oxysuccinimide in dimethylsulfoxide/carbonate suffer, pH 9.0. The solvent was evaporated and the photoheparin was dialyzed against water, and lyophilized, and then dissolved in water at 3 mg/ml. The product is referred to as BBA-EAC-heparin (referring to the benzophenone photoreactive group benzoyl benzoic acid, BBA; and the spacer, epsilon aminocaproic acid, EAC).

Collagenous tissue material (SIS samples) was contacted with biocompatible agent compositions containing photo-heparin (Compound I). The biocompatible agent composition and SIS substrate were treated to bond the biocompatible agent to the SIS substrate. The resulting collagenous tissue material provided acceptable heparin activity over inherent heparin activity of the tissue substrates (i.e., tissue substrates lacking bond heparin).

### Procedure:

The SIS tissue samples were spread into an aluminum foil solution reservoir. After spreading the SIS material, a solution of Compound I (25 mg/ml in water) was poured into each reservoir until the SIS tissue samples were covered in solution. The samples were then subject to irradiation for 1 minute utilizing a Dymax Flood Light (commercially available from Dymax Corporation, Torrington, Conn.). The ultraviolet wand was placed at a distance to provide the samples with approximately 1.5 mW/cm² in the wavelength range of 330-340 nm.

The SIS tissue samples were then flipped and additional Compound I biocompatible agent composition was added to again cover the SIS tissue samples. The tissue samples were subject to irradiation for an additional 1 minute under the conditions noted above.

Tissue samples were then placed in a fresh beaker of distilled water and agitated to remove any unbound Compound I. After soaking for a few minutes, the tissue samples were placed in another fresh beaker of distilled water, agitated, then packaged in a heat-sealed bag in distilled water until use.

The tissue samples were subjected to a Heparin Activity Assay as follows. Prior the Heparin Activity Assay, each tissue sample was removed from the heat-sealed bag, and three small sections from each tissue sample were obtained for the Assay. Two uncoated samples of SIS tissue were used as controls.

### Heparin Activity Assay

The antithrombotic activity of heparin is due to its inhibition of thrombin, which is a protease that is known to participate in the clotting cascade. Heparin inhibits thrombin activity by first binding to antithrombin III (ATIII). The heparin/ATIII complex then binds to and inactivates thrombin, after which the heparin is released and can bind to another ATIII. The assay for inhibition of thrombin by immobilized heparin was conducted by measuring the cleavage of a chromogenic peptide substrate by thrombin.

Each assay was conducted in 1 mL of PBS that contained 0.85 mg BSA (Sigma Chemical Co.), 10 mU human thrombin (Sigma Chemical Co.), 100 mU/mL ATIII (Baxter Biotech, Chicago, Ill.), and 0.17 μmole of the chromogenic thrombin substrate S-2238 (Kabi Pharmacia, Franklin, Ohio). To this assay solution was added either uncoated or heparin coated SIS tissue samples (to evaluate heparin activity on the substrates) or standard concentrations of heparin (to generate standard curves of heparin content versus absorbance). For standard curves, the amounts of heparin that were added ranged from 0 mU to 100 mU. The color generated, measured as absorbance at 405 nm, by thrombin-mediated cleavage of the S-2238 was read using a spectrophotometer after 2 hours of incubation at 37° C. The absorbance was directly related to the activity of the thrombin and, thus, inversely related to the amount of activation of ATIII induced by the heparin in solution or immobilized on the surface of the substrate. Activity of bound heparin was calculated by comparing the absorbance values generated with the membranes to the absorbance values generated with known amounts of added heparin.

Commercial preparations of heparin are commonly calibrated in USP units, 1 unit being defined as the quantity that prevents 1.0 mL of citrated sheep plasma from clotting for 1 hour after the addition of 0.2 mL of 10 g/L CaCl₂ (see Majerus P W, et al. Anticoagulant, thrombolytic, and antiplatelet drugs. In: Hardman J G, Limbrid L E, eds., Goodman and Gilman's The pharmacological bases of therapeutics, 9th ed, New York: McGraw Hill, 1996:1341-6). Commercial preparations of heparin typically include the heparin activity of the preparation. In order to determine the heparin activity of a heparin-treated tissue sample as described herein, the above assay can be performed and compared to a standard generated from a commercial preparation of heparin, based on the above definition of heparin activity.

For all samples, the SIS tissue material had a surface area of 1.43 cm².

TABLE 1
Standards

| | |
|---|---|
| mU Heparin | Absorbance at 405 nm |

| | | |
|---|---|---|
| 0 | 0.763 | 0.754 |
| 10 | 0.608 | 0.598 |
| 20 | 0.417 | 0.417 |
| 30 | 0.336 | 0.346 |
| 40 | 0.284 | 0.283 |
| 50 | 0.260 | 0.263 |
| 66 | 0.213 | 0.214 |
| 100 | 0.165 | 0.168 |

TABLE 2
Results of Heparin Activity Assay.

| | Abs @ | | | |
|---|---|---|---|---|
| Sample | 405 nm | MU | MU/cm² | Average |
| NC #1-1 | 0.764 | 0.158 | 0.110 | 0.0 |
| NC #1-2 | 0.771 | <0 | 0.00 | |
| NC #1-3 | 0.797 | <0 | 0.00 | |
| NC #2-1 | 0.730 | 0.167 | 1.17 | 0.4 |
| NC #2-2 | 0.780 | <0 | 0.00 | |
| NC #2-3 | 0.773 | <0 | 0.00 | |
| 1-1 | 0.475 | 17.0 | 11.9 | 4.9 |
| 1-2 | 0.691 | 3.48 | 2.43 | |
| 1-3 | 0.758 | 0.415 | 0.290 | |
| 2-1 | 0.667 | 4.69 | 3.28 | 4.6 |
| 2-2 | 0.566 | 10.42 | 7.29 | |
| 2-3 | 0.670 | 4.54 | 3.18 | |
| 3-1 | 0.646 | 5.76 | 4.03 | 3.4 |
| 3-2 | 0.714 | 2.41 | 1.68 | |
| 3-3 | 0.636 | 6.29 | 4.40 | |

NC: non-coated, control samples.

According to the results shown in Table 2, heparin activity was detected in connection with SIS tissue samples containing photo-heparin bonded thereto. As much as a ten-fold increase relative to non-coated, control samples was observed for SIS tissue samples provided with biocompatible agent in accordance with aspects of the invention.

### Example 2

### Preparation of Photocollagen

A photoreactive derivative of type IV collagen (photocollagen) is prepared as follows. Human placental type IV collagen is obtained from Sigma Chemical Co., St. Louis, Mo. A heterobifunctional crosslinking agent (BBA-EAC-NOS) is synthesized and used to photoderivatize the collagen.

The BBA-EAC-NOS includes a benzophenone photoreactive group (BBA), a spacer (EAC) and an amine reactive thermochemical coupling group (N-oxysuccinimide, NOS). BBA-EAC is synthesized from 4-benzoylbenzoyl chloride and 6-aminocaproic acid. Then the NOS ester of BBA-EAC is synthesized by esterifying the carboxy group of BBA-EAC by carbodiimide activation with N-hydroxysuccinimide to yield BBA-EAC-NOS.

Type IV collagen is photoderivatized by covalently coupling primary amines on the protein via the NOS ester of BBA-EAC-NOS. The BBA-EAC-NOS is added at a ratio of 10-15 moles of BBA-EAC-NOS per mole of collagen.

### Example 3

### Preparation of Biocompatible Agent including Polymerizable Groups [Collagen Macromer]

A mixture of Types I and II collagen is obtained from Semed-S, Kensey-Nash Corp. The collagen (1.0 grams) is dissolved in 50 mls of 0.01N HCl. When dissolved, 1.25 grams triethylamine (12.4 moles) is added to the reaction mixture. One gram of acryloyl chloride (11.0 mmoles) dissolved in one milliliter of methylene chloride is added to the reaction vessel and the mixture is stirred for 20 hours at room temperature.

The reaction mixture is dialyzed exhaustively against diH₂O, and the product (collagen macromer) isolated by lyophilization.

### Example 4

### Preparation of Polymeric Backbone with Bioactive Groups [BBA-PA-Lysine Reagent]

A polybifunctional reagent comprising polyacrylamide (polymeric backbone) bearing pendent photoreactive groups and pendent lysine (bioactive groups) was prepared as follows.

6-Maleimidohexanoic acid was prepared by dissolving acetic acid in a three-neck, 3 liter flask equipped with an overhead stirrer and drying tube. Maleic anhydride, 78.5 g (0.801 moles), was dissolved in 200 ml of acetic acid and added to the 6-aminohexanoic acid solution. The mixture was stirred one hour while heating on a boiling water bath, resulting in the formation of a white solid. After cooling overnight at room temperature, the solid was collected by filtration and rinsed with 2×50 ml of hexane. Typical yield of the (Z)-4-oxo-5-aza-2-undecendioic acid was 90-95% with a melting point of 160-165° C.

(Z)-4-Oxo-5-aza-2-undecendioic acid, 150.0 g (0.654 moles), acetic anhydride, 68 ml (73.5 g, 0.721 moles), and phenothiazine, 500 mg, were added to a 2 liter three-neck round bottom flask equipped with an overhead stirrer. Triethylamine (TEA), 91 ml (0.653 moles), and 600 ml of tetrahydrofuran (THF) were added and the mixture was heated to reflux while stirring. After a total of 4 hours of reflux, the dark mixture was cooled to <60° C. and poured into a solution of 250 ml of 12 N HCl in 3 liters of water. The mixture was stirred 3 hours at room temperature and then was filtered through a filtration pad (Celite 545, J. T. Baker, Jackson, Tenn.) to remove solids. The filtrate was extracted with 4×500 ml of chloroform and the combined extracts were dried over sodium sulfate. After adding 15 mg of phenothiazine to prevent polymerization, the solvent was removed under reduced pressure. The 6-maleimidohexanoic acid was recrystallized from 2:1 hexane:chloroform to give typical yields of 55-60% with a melting point of 81-85° C.

The 6-Maleimidohexanoic acid, 2.24 g (10.6 mmol) was dissolved in 10.76 g (84.8 mmol) of oxalyl chloride and stirred as a neat solution for 4 hours at room temperature. The excess oxalyl chloride was then removed under reduced pressure and the resulting acid chloride was dissolved in 25 ml of methylene chloride. This solution was added with stirring to a solution of 3.60 g (10.6 mmol) N-ε-t-BOC lysine t-butyl ester hydrochloride (Bachem California) in 25 ml of methylene chloride and 3.21 g (31.7 mmol) of TEA. The resulting mixture was stirred overnight under nitrogen. After this time, the mixture was treated with water and the organic layer was separated and dried over sodium sulfate. The solvent was removed and the product was purified on a silica gel flash chromatography column using a 0-5% methanol in chloroform solvent gradient. Pooling of the desired fractions and evaporation of solvent gave 5.20 g of product (98% yield). Analysis on an NMR spectrometer was consistent with the desired product.

The protected amino acid derivative, 0.566 g (1.14 mmol) was dissolved in 5 ml of trifluoroacetic acid with stirring. After stirring four hours at room temperature, the solvent was removed under reduced pressure. The resulting oil was tritruated with ether to remove residual trifluoroacetic acid to give 373 mg of product for a 98% yield. Analysis on an NMR spectrometer was consistent with the desired product.

Photoreactive groups were then provided to the polymeric backbone as follows. N-[3-(4-Benzoylbenzamido)propyl]methacrylamide (BBA-APMA), the preparation of which is described in Example 3 of U.S. Pat. No. 5,858,653 was utilized to provide the photoreactive group to the reagent. Acrylamide (0.22 g, 3.10 mmol), BBA-APMA (0.014 g, 0.039 mmol), and N-α-[6-(maleimido)hexanoyl]lysine (0.266 g, 0.784 mmol) were dissolved in 7.3 ml of dry DMSO. To initiate the polymerization, 8 mg (0.0407 mmol) of AIBN and 4.0 μl of TEMED were added, followed by spraying with nitrogen to remove all oxygen. The mixture was then heated at 55° C. for 16 hours followed by evaporation of the DMSO under reduced pressure. The product was dissolved in distilled water and dialyzed three days using 6-8K MWCO tubing against distilled water. The resulting solution was lyophilized to give 160 mg of product.

### Example 5

### Preparation of 4-bromomethylbenzophenone (BMBP)

4-Methylbenzophenone (750 g; 3.82 moles) was added to a 5 liter Morton flask equipped with an overhead stirrer and dissolved in 2850 mL of benzene. The solution was then heated to reflux, followed by the dropwise addition of 610 g (3.82 moles) of bromine in 330 mL of benzene. The addition rate was approximately 1.5 mL/min and the flask was illuminated with a 90 watt (90 joule/sec) halogen spotlight to initiate the reaction. A timer was used with the lamp to provide a 10% duty cycle (on 5 seconds, off 40 seconds), followed in one hour by a 20% duty cycle (on 10 seconds, off 40 seconds). At the end of the addition, the product was analyzed by gas chromatography and was found to contain 71% of the desired 4-bromomethylbenzophenone, 8% of the dibromo product, and 20% unreacted 4-methylbenzophenone. After cooling, the reaction mixture was washed with 10 g of sodium bisulfite in 100 mL of water, followed by washing with 3×200 mL of water. The product was dried over sodium sulfate and recrystallized twice from 1:3 toluene:hexane. After drying under vacuum, 635 g of 4-bromomethylbenzophenone was isolated, providing a yield of 60%, having a melting point of 112° C.-114° C. Nuclear magnetic resonance (“NMR”) analysis (¹H NMR (CDCl₃)) was consistent with the desired product: aromatic protons 7.20-7.80 (m, 9 H) and methylene protons 4.48 (s, 2 H). All chemical shift values are in ppm downfield from a tetramethylsilane internal standard.

### Example 6

### Synthesis of Polymeric Backbone with Bioactive Groups [BP-PEG-Lysine Reagent]

A polybifunctional reagent comprising polyethylene glycol (polymeric backbone) bearing pendent benzophenone (“BP,” photoreactive groups) and pendent lysine (bioactive groups) was prepared as follows.

To synthesize benzophenone-tetraethylene glycol (BP-TEG-OH): The tetraethylene glycol (TEG, 77.69 g, 2 molar equivalence) was azeotroped in toluene for two hours. After this time, the remaining toluene was removed in vacuo. The TEG was dissolved in anhydrous THF with stirring under argon at 4° C. Sodium hydride (16.0 g (60%), 2 molar eq.) was washed with hexane and added portionwise. After complete addition, the reaction was stirred for one hour at room temperature. After this time, the BMBP (55.23 g, 0.200 moles, prepared as described in Example 5) was added and the reaction was stirred for sixteen hours at room temperature under an inert atmosphere. The reaction was then quenched with NaCl solution and solvent was removed in vacuo. The resulting residue was dissolved in saturated brine solution, extracted with chloroform and the organic phase was dried over sodium sulfate. The solution was filtered and the solvent was removed in vacuo. The residue was purified by silica flash column chromatography using a gradient solvent system (eluant used was 0% methanol/chloroform to 5% methanol/chloroform) to obtain 11.2 g of 98.8% pure product. Analysis on an NMR spectrometer was consistent with the desired product.

Next, the BP-TEG-OH (11.2 g, 28.8 mmol) and TEA (4.8 ml, 1.2 eq.) were dissolved in 100 mls anhydrous methylene chloride under an inert atmosphere with stirring. The reaction solution was placed on ice and methanesulfonylchloride (2.3 mls, 1.03 eq.) was added with stirring. The reaction was fitted with a drying tube and allowed to warm to room temperature and stirred for 8 hours. After this time, the formed salts were filtered away, the organic phase was washed with a brine solution, and the solvent was removed in vacuo to obtain 11.65 g of product. Analysis on an NMR spectrometer was consistent with the desired product.

A bioactive group, lysine, was added to the polymeric backbone containing photoreactive group as follows. Mesylate (5.1 g, 10.9 mmol) and TEA (7.6 ml, 5 eq.) were added to a 50 ml round bottom flask fitted with a condenser with stirring under an inert atmosphere. The heterogeneous mixture was heated to 80° C. using an oil bath. Next, bioactive group containing a protecting group, H-Lys(Boc)-OtBu.HCl (BACHEM, 4.0 g, 1.1 eq.), was added in two aliquots over 10 minutes to the stirred reaction. As the reaction mixture approached reflux the reaction solution became more homogeneous and was allowed to stir at the elevated temperature for sixteen hours. After this time, the reaction mixture was filtered, washed with cold methylene chloride twice and the solvent was removed in vacuo. The resulting oil was collected and chromatographed using a gradient of 0→5% methanol/chloroform. Appropriate fractions were collected and re-run on similar column using 0→2% methanol/chloroform. Pooling of the desired fractions gave 830 mg of product. Analysis on an NMR spectrometer was consistent with the desired product.

The lysine was deprotected, to obtain a polymeric backbone containing photoreactive groups (BP) and bioactive groups (Lysine) as follows. BP-TEG-Lys(Boc)OtBu (0.83 g, 1.23 mmoles) and trifluoroacetic acid (1.43 mls, 15 eq.) were dissolved in 10 mls methylene chloride with stirring for six hours. After this time, the solvent was removed in vacuo and the product was azeotroped with methylene chloride twice more. The product was dissolved in methylene chloride, washed with 1 N sodium hydroxide followed by brine twice and dried over magnesium sulfate. Solvent was removed in vacuo to give 361 mg of product. Analysis on an NMR spectrometer was consistent with the desired product.

### Example 7

### Lysine Coating on Small Intestine Submucosal Tissue Materials

Substrate: Small intestine submucosal tissue (SIS) was obtained from Cook Biotech Incorporated (West Lafayette, Ind.). The substrates were stored in water at refrigerated temperatures (20° C.) until use.

### Reagents:

Compound II: (Acetylated photo-PVP)

The photoreactive macromer utilized was acetylated photo-PVP. A photoderivatized PVP was prepared as described in U.S. Pat. No. 5,637,460, see Example 4. Generally, the photo-PVP was prepared by copolymerization of 1-vinyl-2-pyrrolidone and N-(3-aminopropyl)methacrylamide (APMA), followed by photoderivatization of the polymer using an acyl chloride (such as, for example, 4-benzoylbenzoyl chloride) under Schotten-Baumann conditions. The acyl chloride reacts with some of the amino groups of the N-(3-aminopropyl) moiety of the copolymer, resulting in the attachment of the aryl ketone to the polymer. The unreated amines of the polymer were acetylated using acetic anhydride to give an acetylated photo-PVP. The liberated hydrochloric acid was neutralized with an aqueous base solution.

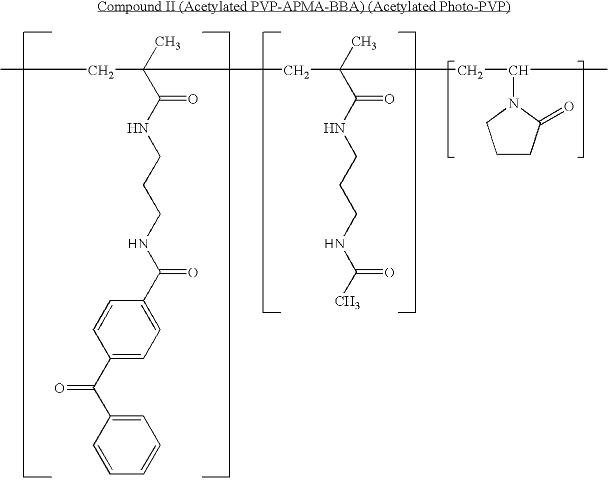

Polybifunctional Reagents:

Reagent A: A polybifunctional reagent comprising polyacrylamide (polymeric backbone) bearing pendent benzophenone (“BP,” photoreactive groups) and pendent lysine (bioactive groups) prepared as described in Example 4. Provided as 10 mg/ml in distilled water.

Reagent B: A polybifunctional reagent comprising tetraethylene glycol (polymeric backbone) bearing pendent benzophenone (“BP,” photoreactive groups) and pendent lysine (bioactive groups) prepared as described in Example 6. Provided as 10 mg/ml in 70% isopropyl alcohol (IPA)/30% water.

Reagent C: A polybifunctional reagent comprising tetraethylene glycol (polymeric backbone) bearing pendent benzophenone (“BP,” photoreactive groups) and pendent lysine (bioactive groups) prepared as described in Example 6. Provided as 10 mg/ml in 30% IPA/70% water.

For Sample II, collagenous tissue material (SIS samples) was contacted first with Compound II, then with polybifunctional Reagent A.

For Samples III, IV and V, collagenous tissue material (SIS samples) were contacted with polybifunctional Reagent A (Sample II), Reagent B (Sample IV) or Reagent C (Sample V).

For all samples, the resulting collagenous tissue material was assayed to determine plasminogen binding from human platelet poor plasma (PPP). The collagenous tissue material prepared provided acceptable plasminogen binding.

### Procedure:

The SIS tissue samples were spread into an aluminum foil reservoir. For Sample II only, after spreading the SIS material, a solution of Compound II (10 mg/ml in water) was poured into each reservoir until the SIS tissue samples were covered in solution. UV cure was performed by illuminating the substrate for one (1) minute utilizing a Dymax Flood Light (commercially available from Dymax Corporation, Torrington Conn.). The ultraviolet wand was placed at a distance to provide the samples with approximately 1.5 mW/cm² in the wavelength range of 330-340 nm.

The SIS tissue samples were then flipped and additional Compound II was added to again cover the SIS tissue samples. The tissue samples were subject to irradiation for an additional one (1) minute under the conditions noted above. The samples were then rinsed one time with distilled water for ten (10) seconds.

For Samples III, IV, and V, tissue samples were contacted with Reagents A, B or C as follows. The SIS tissue samples were spread into an aluminum foil reservoir. After spreading the SIS material, a solution of Reagent A, B or C (as described above) was poured into each reservoir until the SIS tissue samples were covered in solution. UV cure was performed by illuminating the substrate for one (1) minute utilizing a Dymax Flood Light (commercially available from Dymax Corporation, Torrington, Conn.). The ultraviolet wand was placed at a distance to provide the samples with approximately 1.5 mW/cm² in the wavelength range of 330-340 nm. The SIS tissue samples were then flipped and additional Reagent A, B or C, respectively, was added to again cover the SIS tissue samples. The tissue samples were subject to irradiation for an additional one (1) minute under the conditions noted above. The samples were then rinsed one time with distilled water for ten (10) seconds.

The tissue samples were subjected to a Plasminogen Binding Assay as follows. Prior to the Plasminogen Binding Assay, each tissue sample was removed from distilled wtaer and five (5) small sections from each tissue sample were obtained for the Assay. Uncoated samples of SIS tissue were used as controls.

### Plasminogen Binding Assay

The biocompatible activity of lysine is due to its ability to reversibly bind plasminogen from human plasma. Bound plasminogen is cleaved into plasmin, which in turn demonstrates proteolytic activity that cleaves fibrin and prevents fibrin clot formation on a surface. In this Example, the presence of plasminogen bound to tissue samples was observed. Each assay was conducted in 1 ml of PBS that contained human platelet poor plasma (PPP, obtained from George King Biomedical, Inc., Overland Park, Kans.). The PPP was diluted 1:4 in PBS.

To this assay solution was added either uncoated SIS tissue samples, or lysine coated SIS tissue samples. The samples were incubated for 2 hours at room temperature with shaking at 200 rpm. The PPP was aspirated, and the samples were washed three times with 2 ml TRIS-buffered saline with 0.1% Tween-20 (TNT buffer), vortexing briefly. After incubation, the TNT buffer solution was removed.

After the final wash, 1 ml of 1:2000 dilution of anti-human plasminogen goat polyclonal antibody (Cedarlane Laboratories, CL20085A) in phosphate buffered saline (PBS) was added to each test tube, and incubated at room temperature for 30 minutes with shaking at 200 rpm. The antibody solution was then aspirated off each sample, and the samples were rinsed three times in 2 ml of TNT buffer, vortexing briefly.

The rinsed samples were then incubated in 1 ml of 1:6000 dilution of anti-goat-horseradish peroxidase (anti-goat-HRP, Cedarlane Laboratories, CLCC50007) in PBS. The samples were incubated at room temperature for 30 minutes with shaking at 200 rpm. The anti-goat-HRP solution was then aspirated off each sample, and the samples were rinsed five times in 2 ml of TNT buffer.

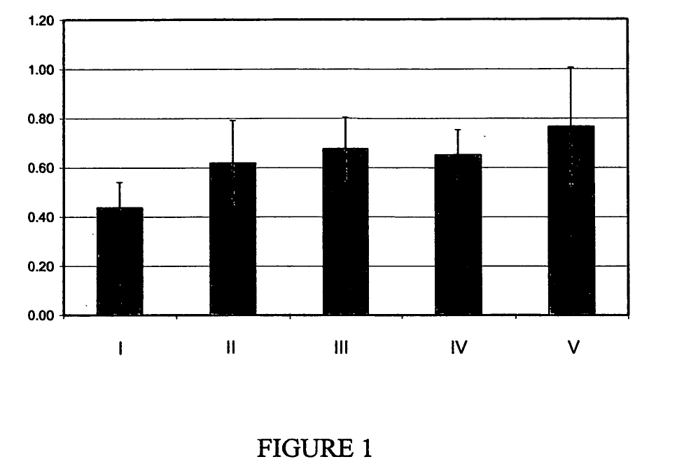

The rinsed samples were then transferred to new test tubes and 1 ml of 1:1 H₂O₂/TMB (3,3′,5,5′ tetramethylbenzidine) solution, which colors in the presence of peroxidase, was added. After the 15-minute incubation, 200 μls of each sample solution was transferred to 4 wells of a 96 well microtiter plate and the absorbance was determined at 650 nm using a spectrophotometer (Molecular Devices, SpectraMax 384 Plus, Sunnyvale, Calif.). Unreacted H₂O₂/TMB was used as a blank. Results are shown in FIG. 1, wherein Sample I is control (uncoated SIS).

### Results:

Single factor ANOVA analysis indicated that the differences among the groups in the experiment as a whole were statistically significant (P<0.05).

When sets of data were compared by two-tailed t tests, all of the coatings except Sample II had significantly more plasminogen binding (P<0.05) than uncoated SIS while there was no clear difference in plasminogen binding between any of the lysine coatings (P>0.05). Average binding on the coated samples (excluding Sample II, since P>0.05) was 49%-75% greater than on the uncoated SIS.

Results indicated that tissue material samples (SIS) containing bound polybifunctional reagents adsorb plasminogen effectively.

Other embodiments of this invention will be apparent to those skilled in the art upon consideration of this specification or from practice of the invention disclosed herein. Variations on the embodiments described herein will become apparent to those of skill in the relevant arts upon reading this description. The inventors expect those of skill to use such variations as appropriate, and intend to the invention to be practiced otherwise than specifically described herein. Accordingly, the invention includes all modifications and equivalents of the subject matter recited in the claims as permitted by applicable law. Moreover, any combination of the above-described elements in all possible variations thereof is encompassed by the invention unless otherwise indicated. All patents, patent documents, and publications cited herein are hereby incorporated by reference as if individually incorporated. In case of conflict, the present specification, including definitions, will control.

What is claimed is:

1. An implantable tissue graft material comprising a collagenous tissue scaffold and a biocompatible agent bonded to the collagenous tissue scaffold via an activated photoreactive group.

2. The implantable tissue graft material according to claim 1 wherein the collagenous tissue scaffold comprises submucosal tissue.

3. The implantable tissue graft material according to claim 2 wherein the submucosal tissue is isolated from intestinal, alimentary, respiratory, urinary or genital tracts of warm-blooded vertebrates, or connective tissue of warm-blooded vertebrates.

4. The implantable tissue graft material according to claim 1 wherein the biocompatible agent is selected from heparin, heparin derivatives, sodium heparin, low molecular weight heparin, hirudin, polylysine, argatroban, glycoprotein IIb/IIIa platelet membrane receptor antibody, coprotein IIb/IIIa platelet membrane receptor antibody, recombinant hirudin, thrombin inhibitor, chondroitin sulfate, modified dextran, albumin, streptokinase, tissue plasminogen activator, and combinations thereof.

5. The implantable tissue graft material according to claim 1 wherein the biocompatible agent is selected from fibronectin, laminin, collagen, elastin, vitronectin, tenascin, fibrinogen, thrombospondin, osteopontin, von Willibrand Factor, bone sialoprotein, hyaluronic acid, chitosan, methyl cellulose, and combinations thereof.

6. The implantable tissue graft material according to claim 1 wherein the biocompatible agent is a polysaccharide.

7. The implantable tissue graft material according to claim 1 wherein the photoreactive group is pendent from the biocompatible agent.

8. The implantable tissue graft material according to claim 1 wherein the photoreactive group is provided by a photoactivatable crosslinking agent.

9. The implantable tissue graft material according to claim 1 wherein the photoreactive group is selected from acetophenone, benzophenone, anthraquinone, anthrone, anthrone-like heterocycles, and substituted derivatives thereof.

10. The implantable tissue graft material according to claim 1 having biocompatible activity that is increased two-fold or more, relative to an inherent biocompatible activity of the collagenous tissue scaffold.

11. The implantable tissue graft material according to claim 4 wherein the biocompatible agent is heparin, and wherein the tissue scaffold has a heparin activity that is at least 5 mU greater than inherent heparin activity of the tissue scaffold.

12. The implantable tissue graft material according to claim 1 formed into an implantable prosthesis.

13. The implantable prosthesis according to claim 12 wherein the prosthesis is tubular, flat, or of a complex shape.

14. A medical product comprising the tissue graft material according to claim 1 provided within sterile medical packaging.

15. A method comprising steps of:
(a) obtaining a tissue graft material comprising a collagenous tissue scaffold;
(b) contacting the collagenous tissue scaffold with a biocompatible agent composition comprising biocompatible agent and one or more photoreactive groups; and
(c) treating the collagenous tissue scaffold and biocompatible agent composition to activate the photoreactive groups and bond the biocompatible agent to the collagenous tissue scaffold via one or more activated photoreactive groups.

16. The method according to claim 15 wherein the step of obtaining a tissue graft material comprises obtaining a cross-linked collagenous tissue scaffold.

17. The method according to claim 15 wherein the one or more photoreactive groups are pendent from the biocompatible agent.

18. The method according to claim 15 wherein the photoreactive group is selected from acetophenone, benzophenone, anthraquinone, anthrone, anthrone-like heterocycles, and substituted derivatives thereof.

19. The method according to claim 15 wherein the contacting step comprises immersing at least a portion of the collagenous tissue scaffold in the biocompatible agent composition.

20. The method according to claim 15 wherein the treating step comprises irradiating the biocompatible agent composition with light in the ultraviolet or visible regions of the spectrum.

21. The method according to claim 15 further comprising a step (d) forming the tissue graft material containing bonded biocompatible agent into an implantable prosthesis.

22. An implantable prosthesis prepared in accordance with the method of claim 15.

23. A method comprising steps of:
(a) obtaining a tissue graft material comprising a collagenous tissue scaffold;
(b) contacting the collagenous tissue scaffold with a reagent having the formula (X)ₘ—Y-Z)ₙ where X is a photoreactive group, Y is a spacer radical, and Z is a bifunctional aliphatic acid; and
(c) treating the collagenous tissue scaffold and biocompatible agent composition to activate the photoreactive groups and bond the reagent to the collagenous tissue scaffold via one or more activated photoreactive groups.

24. An implantable tissue graft material comprising a collagenous tissue scaffold and a reagent having the formula (X)ₘ—Y-Z)ₙ where X is a photoreactive group, Y is a spacer radical, and Z is a bifunctional aliphatic acid or lysine.

25. A method comprising steps of:
(a) obtaining a tissue graft material comprising a collagenous tissue scaffold;
(b) contacting the collagenous tissue scaffold with a reagent comprising a polymeric backbone bearing one or more pendent photoreactive groups and one or more pendent bioactive groups; and
(c) treating the collagenous tissue scaffold and biocompatible agent composition to activate the photoreactive groups and bond the reagent to the collagenous tissue scaffold via one or more activated photoreactive groups,
wherein the bioactive groups are capable of specific, noncovalent interactions with complementary groups when the collagenous tissue scaffold is implanted in a patient.

26. An implantable tissue graft material comprising a collagenous tissue scaffold and a reagent comprising a polymeric backbone bearing one or more pendent photoreactive groups and one or more pendent bioactive groups, wherein the bioactive groups are capable of specific, noncovalent interactions with complementary groups when the collagenous tissue scaffold is implanted in a patient.
